SEQUENCE LISTING (23 sequences)

SEQ ID NO: 1 - protein, 112 aa, synthetic construct
DVVMTQSPLSLPVTPGEPASISCRSSQSLVHSTGNTYLHWYLQKPGQSPQLLIYKVSNRFSGVPDRFSGSGSGTDFTLKISRVEAEDVGVYYCSQSTHVPFTFGGGTKVEIK

SEQ ID NO: 2 - protein, 119 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSS

SEQ ID NO: 3 - protein, 16 aa, synthetic construct
RSSQSLVHSTGNTYLH

SEQ ID NO: 4 - protein, 7 aa, synthetic construct
KVSNRFS

SEQ ID NO: 5 - protein, 9 aa, synthetic construct
SQSTHVPFT

SEQ ID NO: 6 - protein, 10 aa, synthetic construct
GFTFNTYAMY

SEQ ID NO: 7 - protein, 19 aa, synthetic construct
RIRSKSNNYAIYYADSVKD

SEQ ID NO: 8 - protein, 8 aa, synthetic construct
PYSDSFAY

SEQ ID NO: 9 - protein, 16 aa, synthetic construct
RSSLSLVHSTGNTYLH

SEQ ID NO: 10 - protein, 219 aa, synthetic construct
DVVMTQSPLSLPVTPGEPASISCRSSQSLVHSTGNTYLHWYLQKPGQSPQLLIYKVSNRFSGVPDRFSGSGSGTDFTLKISRVEAEDVGVYYCSQSTHVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 449 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 445 aa, synthetic construct
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 112 aa, synthetic construct
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIK

SEQ ID NO: 15 - protein, 111 aa, synthetic construct
QVQLKESGPGLVAPSQSLSITCTVSGFSLSSYGVSWVRQPPGKGLEWLGVIWGDGSTNYKPNLMSRLSISKDISKSQVLFKLNSLQTDDTATYYCVTLDYWGQGTSVTVSS

SEQ ID NO: 16 - protein, 16 aa, synthetic construct
RSSQSLVHRNGNYTLH

SEQ ID NO: 17 - protein, 7 aa, synthetic construct
LVSNRFS

SEQ ID NO: 18 - protein, 9 aa, synthetic construct
FNTTYVPNT

SEQ ID NO: 19 - protein, 6 aa, synthetic construct
SYGVSW

SEQ ID NO: 20 - protein, 21 aa, synthetic construct
VIWGDGSTNYHPNLMSRLSIS

SEQ ID NO: 21 - skipped

SEQ ID NO: 22 - DNA, 336 bp, synthetic construct
  source 1..336: mol_type = other DNA
gatgttgtgatgacccaaactccactctccctgcctgtcagtcttggagatcaagcctccatctcttgcagatctagtcagagccttgtacatagaaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatttgggactttatttctgttttcaaactacatatgttccgaacacgttcggaggggggaccaagctggaaataaaa

SEQ ID NO: 23 - DNA, 333 bp, synthetic construct
  source 1..333: mol_type = other DNA
caggtgcagctgaaggagtcaggacctggcctggtggcgccctcacagagcctgtccatcacatgcactgtctcagggttctcattaagcagctatggtgtaagctgggttcgccagcctccaggaaagggtctggagtggctgggagtaatatggggtgacgggagcacaaattatcatccaaatctcatgtccagactgagtatcagcaaggatatttccaagagccaagttctcttcaaactgaatagtctgcaaactgatgacacagccacgtactactgtgtcaccttcgactactggggtcaaggaacctcagtcaccgtctcctca

(19) United States
(12) Patent Application Publication
(10) Pub. No.: US 2025/0345418 A1
(43) Pub. Date: Nov. 13, 2025

(54) METHODS OF TREATING AL AMYLOIDOSIS

(71) Applicant: Prothena Biosciences Limited, Dublin (IE)

(72) Inventors: Gene Kinney, Boca Raton, FL (US); Wagner Zago, San Carlos, CA (US); Radhika Tripuraneni, Menlo Park, CA (US); Carol Karp, Hillsborough, CA (US)

(21) Appl. No.: 18/727,991

(22) PCT Filed: Jan. 11, 2023
(86) PCT No.: PCT/US2023/060503

§ 371 Date: Jul. 10, 2024

Related U.S. Application Data
(60) Provisional application No. 63/298,396, filed on Jan. 11, 2022.

(51) Int. Cl. A61K 39/395; A61K 39/00; C07K 16/18; C07K 16/28

(52) U.S. Cl. CPC A61K 39/3955; C07K 16/18; C07K 16/2896; A61K 2039/507; A61K 2039/545; C07K 2317/24

(57) ABSTRACT

Antibody formulations and methods useful for treatment of patients with AL amyloidosis.

### CROSS-REFERENCE TO RELATED APPLICATIONS

This application is the National Stage of International Application No. PCT/US2023/060503, filed on Jan. 11, 2023, which claims priority to U.S. Provisional Patent Application No. 63/298,396, filed on Jan. 11, 2022. The contents of these applications are incorporated herein by reference in their entirety.

### SEQUENCE LISTING

This application contains a Sequence Listing that has been submitted electronically as an XML file named “50887-0021US1_SL_ST26.XML.” The XML file, created on Dec. 30, 2022, is 22,879 bytes in size. The material in the XML file is hereby incorporated by reference in its entirety.

### FIELD

The disclosure relates to the technical fields of immunology and medicine.

### BACKGROUND

AL amyloidosis involves a hematological disorder caused by clonal plasma cells that produce immunoglobulin light chains that can misfold and contribute to disease. Overproduction of misfolded light chain by plasma cells results in deposits of abnormal AL protein (amyloid) in the tissues and organs of individuals with AL amyloidosis. Clinical features of AL amyloidosis include a constellation of symptoms and organ dysfunction that can include cardiac, renal, and hepatic dysfunction, gastrointestinal involvement, neuropathies and macroglossia. The mechanisms by which amyloidogenic immunoglobulin light chains result in organ dysfunction are not well characterized, however, it is hypothesized that both amyloid deposits and prefibrillar aggregates may contribute to cytotoxic effects on organs observed in patients with AL amyloidosis. AL amyloidosis is a disease entity of its own, although AL amyloidosis can occur concurrently in a subset of patients with multiple myeloma (up to 15%) or monoclonal gammopathy of unknown significance (MGUS; up to 9%).

AL amyloidosis is a rare disorder with an estimated incidence of 8 in 1,000,000 people. Only 1200 to 3200 new cases of AL amyloidosis are reported each year in the United States. Two thirds of patients with AL amyloidosis are male and less than 5% of patients are under 40 years of age. Both the causes and origins of AL amyloidosis remain poorly understood. The outcome of the disease for patients with AL amyloidosis can be predicted based on the Mayo four stage prognostic staging system discussed in Kumar et al., 2012 (Kumar et al., Revised Prognostic Staging System for Light Chain Amyloidosis Incorporating Cardiac Biomarkers and Serum Free Light Chain Measurements, J Clin Oncol 30:989-995 2012), with the outcome for Stage IV patients being quite dire.

Current treatment of patients with AL amyloidosis is aimed at reducing or eliminating the bone marrow disorder, i.e. the plasma cells that are responsible for producing the light chains, thereby limiting or halting the production of amyloid. The most aggressive treatment options include stem cell transplant and high-dose chemotherapy for those patients who can tolerate it. Other treatment regimens include combinations of drugs often used to treat hematological malignancies, such as melphalan, prednisone, dexamethasone and proteosome inhibitors such as bortezomib, in an attempt to reduce light chain production. There are no currently approved treatments for AL amyloidosis that directly target potentially toxic forms of the amyloidogenic proteins. While some treatment options may ameliorate some of the morbidity associated with AL amyloidosis, few if any have been demonstrated to improve the prognosis in patients.

In 2021, FDA approved the use of subcutaneous daratumumab in combination with cyclophosphamide-bortezomib-dexamethasone (CyBorD) for the treatment of newly diagnosed AL amyloidosis patients with Mayo stages I to IIIA (https://www.fda.gov/drugs/drug-approvals-and-databases/fda-grants-accelerated-approval-darzalex-faspro-newly-diagnosed-light-chain-amyloidosis). Despite this, no clear survival benefit has been observed for patients with AL amyloidosis treated with daratumumab. At median follow-up time of 11.4 months in the Phase 3 ANDROMEDA study, 56 patients died-27 in the daratumumab group (daratumumab+CyBorD) and 29 in the control group (CyBorD). Kastritis et al. N Engl J Med. 2021 Jul. 1;385 (1): 46-58.

Furthermore, Mayo Stage IV patients with AL amyloidosis represent a patient subset with a very high burden of morbidity and mortality, with no currently approved treatments, and population estimates of approximately 2,760 patients in the U.S., and from 6,900 to 10,350 patients in the U.S. and the European Union combined.

Thus, there is an unmet need for therapies that improve health status, including reducing the risk of mortality or enhancing the quality of life in patients with AL amyloidosis.

### SUMMARY

The present disclosure relates to methods of treating certain AL amyloidosis patients. Provided herein are methods of treating a patient having AL amyloidosis, including: administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient; and 5 to 90 minutes after completion of administering the antibody, administering a therapeutically effective dosage of daratumumab to the patient, where the patient has (a) Mayo Stage IV AL amyloidosis.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters. In some embodiments, the therapeutically effective dosage of daratumumab to the patient is administered approximately 60 minutes after completion of administering the antibody.

Also, provided herein are methods of treating a patient having AL amyloidosis, including:
(a) determining one or more of the following:
   (i) the Mayo Stage of the patient's AL amyloidosis;
   (ii) the 6 minute walk test distance (6MWT) of the patient;
   (iii) the ejection fraction (EF) of the patient; or
   (iv) cardiac involvement of the patient;
(b) selecting the patient for treatment with (i) an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), and (ii) daratumumab, if the patient has Mayo Stage IV amyloidosis;
(c) administering an effective dosage of the antibody to the selected patient; and
(d) 5 to 90 minutes after completion of administering the antibody, administering a therapeutically effective dosage of daratumumab to the selected patient.

In some embodiments, the selected patient has a 6MWT≥150 meters and an EF>50%. In some embodiments, the selected patient has a 6MWT≥30 meters and ≤550 meters. In some embodiments, the selected patient has an EF>50%. In some embodiments, the selected patient has a 6MWT≥150 meters. In some embodiments, the selected patient has a 6MWT≥30 meters and ≤550 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

In some embodiments, the therapeutically effective dosage of daratumumab to the patient is administered approximately 60 minutes after completion of administering the antibody.

Also provided herein are methods of treating a patient having Mayo Stage IV AL amyloidosis and having NYHA class III or NYHA class IV, the method including: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

In some embodiments, the patient's NYHA class is reduced by at least two classes. In some embodiments, the patient's NYHA class is assessed nine or more months after treatment.

Also provided herein are methods of reducing mortality or the risk of mortality in a patient having Mayo Stage IV AL amyloidosis and having NYHA class III or NYHA class

IV, the method including: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the risk of mortality is all-cause mortality. In some embodiments, the risk of mortality is cardiac mortality. In some embodiments, the risk of mortality is assessed nine or more months after treatment. In some embodiments, the risk of mortality is reduced by at least 15% as compared to the risk of mortality in a control population. In some embodiments, mortality is reduced within 8 months after treatment. In some embodiments, mortality is reduced within 9 months after treatment. In some embodiments, mortality is reduced within 10 months after treatment. In some embodiments, mortality is reduced within 11 months after treatment. In some embodiments, mortality is reduced within 12 months after treatment.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of reducing the risk of hospitalization in a patient having Mayo Stage IV AL amyloidosis, the method including: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥ 30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the risk of hospitalization in the patient is reduced by at least 20% as compared to the risk of hospitalization in a control population.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of increasing the number of days alive out of hospital (DAOH) in a patient Mayo Stage IV amyloidosis, the method including: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the number of DAOH increases by at least 20 days after one month of treatment as compared to a control population. In some embodiments, the number of DAOH increases by at least 100 days after six months of treatment as compared to DAOH in a control population. In some embodiments, the number of DAOH increases by at least 300 after twelve months of treatment as compared to DAOH in a control population. In some embodiments, the number of DAOH increases by at least 600 after 24 months of treatment as compared to DAOH in a control population. In some embodiments, the number of DAOH increases by at least 900 after 48 months of treatment as compared to DAOH in a control population.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement.

In some embodiments, the patient has Mayo Stage IV AL amyloidosis, cardiac involvement, and a 6MWT≥30 meters and ≤550 meters.

Also provided herein are methods of reducing NTproBNP level in a patient having Mayo Stage IV AL amyloidosis, the method including: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the NTproBNP level is reduced by at least 31% from a baseline after 3 months of treatment. In some embodiments, the NTproBNP level is reduced by at least 45% from a baseline after 3 months of treatment. In some embodiments, the NTproBNP level is reduced by at least 60% from a baseline after 3 months of treatment. In some embodiments, the NTproBNP level is reduced by at least 60% from a baseline to a nadir>400 μg/ml after 3 months of treatment. In some embodiments, the NTproBNP level is reduced to a nadir<400 μg/ml after 3 months of treatment. In some embodiments, the NTproBNP level is reduced by at least 45% from a baseline after 6 months of treatment. In some embodiments, the NTproBNP level is reduced by at least 60% from a baseline after 6 months of treatment. In some embodiments, the NTproBNP level is reduced by at least 60% from a baseline to a nadir>400 μg/ml after 6 months of treatment. In some embodiments, the NTproBNP level is reduced to a nadir<400 μg/ml after 3 months of treatment.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement.

In some embodiments, the patient has Mayo Stage IV AL amyloidosis, cardiac involvement, and a 6MWT≥30 meters and ≤550 meters.

Also provided herein are methods of improving cardiac response rate in a patient having Mayo Stage IV AL amyloidosis, the method including: administering a therapeutically effective amount of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chainimmunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the cardiac response rate increases at least 30% after 6 months of treatment as compared to baseline. In some embodiments, the cardiac response rate increases at least 50% after 12 months of treatment as compared to baseline. In some embodiments, the cardiac response rate increases at least 75% after 12 months of treatment as compared to baseline.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of improving a six minute walk test (6MWT) in a patient having Mayo Stage IV AL amyloidosis, the method including administering a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the 6MWT achieved is at least 300 meters. In some embodiments, the 6MWT achieved is at least 300 meters after treatment for 3 months. In some embodiments, the 6MWT achieved is at least 300 meters after treatment for 6 months. In some embodiments, the 6MWT achieved is at least 300 meters after treatment for 9 months. In some embodiments, the 6MWT achieved is at least 300 meters after treatment for 12 months. In some embodiments, the 6MWT achieved is at least 300 meters after treatment for 15 months. In some embodiments, the 6MWT achieved is at least 300 meters after treatment for 18 months.

In some embodiments, the 6MWT improves at least 33 metersascompared to baseline. In some embodiments, the 6MWT improves at least 33 meters after 3 months of treatment compared to baseline. In some embodiments, the 6MWT improves at least 33 meters after 6 months of treatment compared to baseline. In some embodiments, the 6MWT improves at least 33 meter after 9 months of treatment compared to baseline. In some embodiments, the 6MWT improves at least 33 meters after 12 months of treatment as compared to baseline. In some embodiments, the 6MWT improves at least 33 meters after 15 months of treatment. In some embodiments, the 6MWT improves at least 33 meters after 18 months of treatment. In some embodiments, the 6MWT achieved is at least 300 meters after treatment for 18 months. In some embodiments, the 6MWT improves at least 33 meters after 18 months of treatment as compared to baseline.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of increasing a Kansas City Cardiomyopathy Questionnaire (KCCQ) score in a patient having Mayo Stage IV AL amyloidosis, the method including: administering a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the KCCQ score increased by at least 5 after 3 months of treatment as compared to baseline. In some embodiments, the KCCQ score increased by at least 10 after 3 months of treatment as compared to baseline. In some embodiments, the KCCQ score increased by at least 15 after 3 months of treatment as compared to baseline. In some embodiments, the KCCQ score improves by at least 20 after 3 months of treatment as compared to baseline. In some embodiments, the KCCQ score improves by at least 5 after 12 months of treatment as compared to baseline. In some embodiments, the KCCQ score improves by at least 10 after 12 months of treatment as compared to baseline. In some embodiments, the KCCQ score improves by at least 15 after 12 months of treatment as compared to baseline. In some embodiments, the KCCQ score improves by at least 20 after 12 months of treatment as compared to baseline.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of increasing Major Organ Deterioration Progression Free Survival in a patient having Mayo Class IV AL amyloidosis, the method including: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the NTproBNP level is reduced by at least 31% from a baseline after 3 months of treatment. In some embodiments, the NTproBNP level is reduced by at least 45% from a baseline after 3 months of treatment. In some embodiments, the NTproBNP level is reduced by at least 60% from a baseline after 3 months of treatment. In some embodiments, the NTproBNP level is reduced by at least 60% from a baseline to a nadir>400 μg/ml after 3 months of treatment. In some embodiments, the NTproBNP level is reduced to a nadir<400 μg/ml after 3 months of treatment. In some embodiments, the NTproBNP level is reduced by at least 45% from a baseline after 6 months of treatment. In some embodiments, the NTproBNP level is reduced by at least 60% from a baseline after 6 months of treatment. In some embodiments, the NTproBNP level is reduced by at least 60% from a baseline to a nadir>400 μg/ml after 6 months of treatment. In some embodiments, the NTproBNP level is reduced to a nadir<400 μg/ml after 3 months of treatment.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of treating a patient with AL amyloidosis, including: (a) determining that the patient has a 6 minute walk distance (6MWT)≥30 meters and ≤550 meters or a 6MWT≥150 meters and/or an ejection fraction (EF)>50%; (b) selecting the patient for treatment with an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), and daratumumab; (c) administering an effective dosage of the antibody to the selected patient; and (d) 5 to 90 minutes after completion of administering the antibody, administering a therapeutically effective amount of daratumumab to the selected patient, wherein the patient has one or more of the following: (a) Mayo Stage IV AL amyloidosis, (b) a 6 minute walk distance (6MWT)≥30 meters and ≤550 meters, (c) a 6MWT≥150 meters and EF>50%, (d) Mayo Stage IV and an EF>50%, (e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥ 30 meters and ≤550 meters.

In some embodiments, the patient has Mayo Stage IV AL amyloidosis. In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of treating a patient with AL amyloidosis who has a demonstrated 6 minute walk distance (6MWT) of ≥30 meters and ≤550 meters or a 6MWT greater than or equal to 150 meters and/or an ejection fraction (EF) of more than 50%, including: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105; and 5 to 90 minutes after completion of administering the antibody, administering a therapeutically effective amount of daratumumab to the patient, wherein the patient has one or more of the following: (a) Mayo Stage IV AL amyloidosis, (b) a 6 minute walk distance (6MWT)≥30 meters and ≤550 meters, (c) a 6MWT≥150 meters and EF>50%, (d) Mayo Stage IV and an EF>50%, (e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥30 meters and ≤550 meters.

In some embodiments, the patient has Mayo Stage IV AL amyloidosis. In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of reducing the risk of mortality in a patient with AL amyloidosis by at least 45%, including: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), 6MWT and 5 to 90 minutes after completion of administering the antibody, administering a therapeutically effective amount of daratumumab to the patient, wherein the patient has one or more of the following: (a) Mayo Stage IV AL amyloidosis, (b) a 6 minute walk distance (6MWT)≥30 meters and ≤550 meters, (c) a 6MWT≥150 meters and EF>50%, (d) Mayo Stage IV and an EF>50%, (e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥30 meters and ≤550 meters.

In some embodiments, the risk of mortality is of all-cause mortality. In some embodiments, the risk of all-cause mortality is reduced by at least about 48.9%. In some embodiments, the risk of all-cause mortality is reduced by at least about 50%. In some embodiments, the risk of all-cause mortality is reduced by at least about 50.2%. In some embodiments, the risk of all-cause mortality is reduced by at least about 60%. In some embodiments, the risk of all-cause mortality is reduced by at least about 70%. In some embodiments, the risk of all-cause mortality is reduced by at least about 79.9%. In some embodiments, the risk of all-cause mortality is reduced by at least about 81.5%.

In some embodiments, risk of mortality is of cardiac mortality. In some embodiments, the risk of cardiac mortality is reduced by at least about 75%. In some embodiments, the risk of cardiac mortality is reduced by at least about 62.2%.

In some embodiments, the patient has Mayo Stage IV AL amyloidosis. In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Also provided herein are methods of reducing global longitudinal strain (GLS) in a patient with AL amyloidosis including: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), 6MWT and 5 to 90 minutes after completion of administering the antibody, administering a therapeutically effective amount of daratumumab to the patient, wherein the patient has one or more of the following: (a) Mayo

Stage IV AL amyloidosis, (b) a 6 minute walk distance (6MWT)≥30 meters and ≤550 meters, (c) a 6MWT≥150 meters and EF>50%, (d) Mayo Stage IV and an EF>50%, (e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥30 meters and ≤550 meters.

In some embodiments, the administering results in a reduction of GLS of about 0.1% to about 10% in the patient. In some embodiments, the administering results in a reduction of GLS of about 0.5% to about 10% in the human subject. In some embodiments, the administering results in a reduction of GLS of about 1% to about 10% in the human subject. In some embodiments, the administering results in a reduction of GLS of at least about 0.5% in the human subject. In some embodiments, the administering results in a reduction of GLS of at least about 1.0% in the human subject. In some embodiments, the administering results in a reduction of GLS of at least about 2.0% in the human subject. In some embodiments, the administering results in a reduction of GLS of at least about 10% in the human subject.

In some embodiments, the patient has Mayo Stage IV AL amyloidosis. In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of improving SF-36v2 physical component summary (PCS) score in a patient with AL amyloidosis including: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), and 5 to 90 minutes after completion of administering the antibody, administering a therapeutically effective amount of daratumumab to the patient, where the SF-36v2 score is determined at least 1 months after treatment, wherein the patient has one or more of the following: (a) Mayo Stage IV AL amyloidosis, (b) a 6 minute walk distance (6MWT)≥30 meters and ≤550 meters, (c) a 6MWT≥150 meters and EF>50%, (d) Mayo Stage IV and an EF>50%, (e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥30 meters and ≤550 meters . . .

In some embodiments, the duration of treatment is effective to achieve or maintain at least about a 5 point increase from baseline in SF-36v2. In some embodiments, the SF-36v2 PCS score is determined after 2 months of treatment. In some embodiments, the SF-36v2 PCS score is determined after 3 months of treatment. In some embodiments, the SF-36v2

PCS score is determined after 6 months of treatment. In some embodiments, the SF-36v2 PCS score is determined after 9 months of treatment. In some embodiments, the SF-36v2 PCS score is determined after 12 months of treatment.

In some embodiments, the patient has Mayo Stage IV AL amyloidosis. In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of reducing mortality or the risk of mortality in a patient having Mayo Stage IV AL amyloidosis, including: administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient; and 5 to 90 minutes after completion of administering the antibody, administering a therapeutically effective dosage of daratumumab to the patient.

In some embodiments, the risk of mortality is all-cause mortality. In some embodiments, the risk of mortality is cardiac mortality.

In some embodiments, the risk of mortality is reduced after 1 month of treatment. In some embodiments, the risk of mortality is reduced after 2 months of treatment. In some embodiments, the risk of mortality is reduced after 3 months of treatment. In some embodiments, the risk of mortality is reduced after 4 months of treatment. In some embodiments, the risk of mortality is reduced after 5 months of treatment. In some embodiments, the risk of mortality is reduced after 6 months of treatment. In some embodiments, the risk of mortality is reduced after 7 months of treatment. In some embodiments, the risk of mortality is reduced after 8 months of treatment. In some embodiments, the risk of mortality is reduced after 9 months of treatment. In some embodiments, the risk of mortality is reduced after 10 months of treatment. In some embodiments, the risk of mortality is reduced after 11 months of treatment. In some embodiments, the risk of mortality is reduced after 12 months of treatment.

In some embodiments, the risk of mortality is reduced by at least 15% as compared to the risk of mortality in a control population.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of reducing global longitudinal strain (GLS) in a patient having Mayo Stage IV AL amyloidosis, including administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient.

In some embodiments, the administering results in a reduction of GLS of about 0.1% to about 10% in the patient. In some embodiments, the administering results in a reduction of GLS of about 0.5% to about 10% in the human subject. In some embodiments, the administering results in a reduction of GLS of about 1% to about 10% in the human subject. In some embodiments, the administering results in a reduction of GLS of at least about 0.5% in the human subject. In some embodiments, the administering results in a reduction of GLS of at least about 1.0% in the human subject. In some embodiments, the administering results in a reduction of GLS of at least about 2.0% in the human subject. In some embodiments, the administering results in a reduction of GLS of at least about 10% in the human subject.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also provided herein are methods of reducing global longitudinal strain (GLS) in a patient having Mayo Stage IV AL amyloidosis, including: administering an effective dosage of an antibody which competes for binding to human amyloid A peptide or human kappa or lambda light chain immunoglobulin with 2A4 (ATCC Accession Number PTA-9662) or 7D8

(ATCC Accession Number PTA-9468), or competes for binding to kappa light chain immunoglobulin with 11-1F4 (ATCC Accession Number PTA-105) to the patient; and 5 to 90 minutes after completion of administering the antibody, administering a therapeutically effective dosage of daratumumab to the patient.

In some embodiments, the administering results in a reduction of GLS of about 0.1% to about 10% in the patient. In some embodiments, the administering results in a reduction of GLS of about 0.5% to about 10% in the human subject. In some embodiments, the administering results in a reduction of GLS of about 1% to about 10% in the human subject. In some embodiments, the administering results in a reduction of GLS of at least about 0.5% in the human subject. In some embodiments, the administering results in a reduction of GLS of at least about 1.0% in the human subject. In some embodiments, the administering results in a reduction of GLS of at least about 2.0% in the human subject. In some embodiments, the administering results in a reduction of GLS of at least about 10% in the human subject.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement. In some embodiments, the patient has Mayo Stage IV AL amyloidosis, cardiac involvement, and a 6MWT≥30 meters and ≤550 meters.

Also, provided herein are methods of treating a patient having AL amyloidosis, including: administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient; and 5 to 90 minutes after completion of administering the antibody, administering a therapeutically effective dosage of daratumumab to the patient, where the patient has (a) Mayo Stage IV AL amyloidosis.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters. In some embodiments, the therapeutically effective dosage of daratumumab to the patient is administered approximately 60 minutes after completion of administering the antibody.

In some embodiments, the antibody includes a light chain variable region including three complementarity determining regions set forth as SEQ ID NOs: 3, 4 and 5, or SEQ ID NOs: 16, 17, and 18, and a heavy chain variable region including three complementarity determining regions set forth as SEQ ID NOs: 6, 7 and 8, or SEQ ID NOs: 19, 20, and 21. In some embodiments, the light chain variable region includes the amino acid sequence set forth as SEQ ID NO: 1 or 14. In some embodiments, the heavy chain variable region includes the amino acid sequence set forth as SEQ ID NO: 2 or 15. In some embodiments, the light chain variable region includes the amino acid sequence set forth as SEQ ID NO: 1 or 14, and the heavy chain variable region includes the amino acid sequence set forth as SEQ ID NO: 2 or 15. In some embodiments, the antibody includes a light chain including the amino acid sequence set forth as SEQ ID NO: 10, and a heavy chain including the amino acid sequence set forth as SEQ ID NO: 11, 12 or 13. In some embodiments, the antibody includes a light chain including the amino acid sequence set forth as SEQ ID NO: 10, and a heavy chain including the amino acid sequence set forth as SEQ ID NO: 12.

In some embodiments, the patient previously received or concomitantly receives treatment with melphalan, prednisone, dexamethasone, bortezomib, cyclophosphamide, lenalidomide, doxorubicin, or a combination thereof.

In some embodiments, the antibody includes a light chain variable region including three complementarity determining regions of 2A4, 7D8 or 11-1F4, and a heavy chain variable region including three complementarity determining regions of 2A4, 7D8 or 11-1F4, respectively. In some embodiments, the antibody is a humanized version of 2A4. In some embodiments, the antibody is a humanized or chimeric version of 11-1F4. In some embodiments, the antibody is birtamimab.

In some embodiments, the patient exhibits improvement in the 36-Item Short Form Survey Physical Component Score (SF-36 PCS) or SF-36v2 following treatment with the antibody. In some embodiments, after nine months of treatment the change in the patient's score on the SF-36 PCS or SF-36v2 is at least 5 points higher relative to a different patient at the same time point who has not been administered the antibody.

In some embodiments, the therapeutically effective dosage of the antibody is administered from a pharmaceutical formulation including the antibody at a concentration within the range from about 1 mg/mL to about 100 mg/mL. In some embodiments, the therapeutically effective dosage of the antibody is from about 0.5 mg/kg to about 30 mg/kg and the antibody is administered intravenously or subcutaneously at a frequency of from about weekly to about quarterly. In some embodiments, the therapeutically effective dosage of the antibody is present at a concentration of about 50 mg/mL.

In some embodiments, the therapeutically effective dosage is administered intravenously following the transfer of an amount of the formulation required for the dosage from a vial to an intravenous bag containing a liquid. In some embodiments, the therapeutically effective dosage is about 24 mg/kg and the antibody is administered intravenously every 28 days.

In some embodiments, the duration of the treatment is at least 15 months. In some embodiments, the duration of the treatment is at least 12 months. In some embodiments, the duration of the treatment is at least 9 months. In some embodiments, the duration of the treatment is at least 6 months. In some embodiments, the duration of the treatment is at least 3 months.

In some embodiments, the duration is effective to achieve or maintain at least about a 3 point increase from baseline in SF-36 PCS or SF-36v2.

In some embodiments, the antibody is a Fab, Fab′, F(ab′) 2, F (ab) c, Dab, nanobody, or Fv.

In some embodiments, treatment results in a 20% relative reduction in hospitalization. In some embodiments, treatment results in a 31% to 60% reduction in NTproBNP from a baseline. In some embodiments, treatment results in a>60% reduction in the NTproBNP level from a baseline to a nadir NTproBNP>400 μg/mL. In some embodiments, treatment results in a nadir NTproBNP level of <400 μg/mL.

In some embodiments, treatment results in at least a 5 point change to a 20 point change in the Kansas City Cardiomyopathy Questionnaire (KCCQ) score.

In some embodiments, after treatment of 3 months results in at least a 33 meter improvement in the 6MWT. In some embodiments, after treatment of 6 months results in at least a 33 meter improvement in the 6MWT. In some embodiments, after treatment of 9 months results in at least a 33 meter improvement in the 6MWT. In some embodiments, after treatment of 12 months results in at least a 33 meter improvement in the 6MWT. In some embodiments, after treatment of 15 months results in at least a 33 meter improvement in the 6MWT. In some embodiments, after treatment of 18 months results in at least a 33 meter improvement in the 6MWT.

In some embodiments, if the patient has an NTproBNP level≥8500 μg/mL, the patient is excluded from receiving treatment.

In some embodiments, the patient did not receive an autologous transplant prior to administration of the effective dosage of the antibody. In some embodiments, the patient cannot receive an autologous transplant during treatment.

In some embodiments, daratumumab is administered 120 minutes after completion of administering the antibody. In some embodiments, daratumumab is administered 90 minutes after completion of administering the antibody. In some embodiments, daratumumab is administered 75 minutes after completion of administering the antibody. In some embodiments, daratumumab is administered 60 minutes after completion of administering the antibody. In some embodiments, daratumumab is administered 30 minutes after completion of administering the antibody. In some embodiments, daratumumab is administered 15 minutes after completion of administering the antibody. In some embodiments, daratumumab is administered 5 minutes after completion of administering the antibody.

In some embodiments, the patient meets the following criteria: (i) past documented or presently noted clinical signs and symptoms supportive of a diagnosis of heart failure in the setting of a confirmed diagnosis of AL amyloidosis in the absence of an alternative explanation for heart failure; and (ii) an endomyocardial biopsy demonstrating AL amyloidosis or an echocardiogram demonstrating a mean left ventricular wall thickness at diastole>12 mm in the absence of other causes. In some embodiments, the other causes include severe hypertension or aortic stenosis.

### DETAILED DESCRIPTION

The disclosure provides methods of treating certain AL amyloidosis patients, namely patients with Mayo Stage IV AL amyloidosis. In some embodiments, the Mayo Stage IV AL amyloidosis patients have a baseline six minute walk distance (6MWT; sometimes referred to as the six minute walk test (6MWT) distance) greater than or equal to 150 meters, Mayo Stage IV patients with a baseline EF greater than 50%, Mayo Stage IV patients with a baseline 6MWT greater than or equal to 150 meters and ejection fraction (EF) greater than 50%, has cardiac involvement; has Mayo Stage IV AL amyloidosis and has cardiac involvement; and/or has Mayo Stage IV AL amyloidosis, cardiac involvement, and a 6MWT≥30 meters and ≤550 meters. In some embodiments, the patient has an ejection fraction greater than 50%. The methods involve administering to such patients an antibody which competes for binding to human amyloid A peptide or human kappa or lambda light chain immunoglobulin with antibody 2A4 (ATCC Accession Number PTA-9662) or antibody 7D8 (ATCC Accession Number PTA-9468) or which competes for binding to kappa immunoglobulin light chain with antibody 11-1F4 (ATCC Accession Number PTA-150) and to 90 minutes after administering the antibody, administering a therapeutically effective dosage of daratumumab to the patient, wherein the patient has Mayo Stage IV AL amyloidosis. The Mayo Stage IV amyloidosis patients can also have: a 6MWT≥30 meters and ≤550 meters; a 6MWT≥150 meters and an EF>50% at baseline; Mayo Stage IV and a 6MWT≥30 meters and ≤550 meters; Mayo Stage IV and EF>50% at baseline; Mayo Stage IV, a 6MWT≥150 meters and an EF>50% at baseline; has cardiac involvement; Mayo 10 Stage IV AL amyloidosis and has cardiac involvement; or Mayo Stage IV AL amyloidosis, cardiac involvement, and a 6MWT≥30 meters and ≤550 meters. In some embodiments, the antibody is birtamimab.

### I. Definitions

The term “antibody” includes intact antibodies and antigen-binding fragments thereof. Typically, fragments compete with the intact antibody from which they were derived for specific binding to the target including separate heavy chains, light chains Fab, Fab′, F(ab′) 2, F (ab) c, Dabs, nanobodies, and Fv. Fragments can be produced by recombinant DNA techniques, or by enzymatic or chemical separation of intact immunoglobulins. The term “antibody” also includes a bispecific or multispecific antibody and/or a humanized antibody. 20 A bispecific or bifunctional or multifunctional antibody is an artificial hybrid antibody having two or more different heavy/light chain pairs and two or more different binding sites (see, e.g., Songsivilai and Lachmann, (lin. Exp. Immunol., 79:315-321 (1990); Kostelny et al., J. Immunol., 148:1547-53 (1992)).

The term “baseline” includes an initial measurement of a condition that is taken at an early time point and used for comparison over time to look for changes and which is used for comparison with later data (e.g., such as response to treatment as measured by scores, tests, or other described measurement techniques described herein).

The term “censoring” refers to a situation in which the value of a measurement or observation is only partially known. For example, if a study is conducted to measure the impact of a drug on mortality rate, survival will be assumed for the period of the study in the absence of data indicating death, such that patients who withdrew from the study are considered to be alive through the duration of the study regardless of their unknown disposition (i.e., alive or dead).

The term “control population” or “control group” includes a population or group of patients that can receive a placebo, another treatment, or do not receive a drug or treatment at all (e.g., treatment with any of the antibodies described herein). The patients receiving treatment can be compared to the control population or control group.

The term “cardiac involvement” refers to past documented or presently noted clinical signs and symptoms supportive of a diagnosis of heart failure in the setting of a confirmed diagnosis of AL amyloidosis in the absence of an alternative explanation for heart failure and either an endomyocardial biopsy demonstrating AL amyloidosis or an echocardiogram demonstrating a mean left ventricular wall thickness at diastole>12 mm in the absence of other causes (e.g., severe hypertension, aortic stenosis), which would adequately explain the degree of wall thickening.

The term “ejection fraction” or “EF” refers to the measurement of how much blood the left ventricle pumps out with each contraction. An ejection fraction of 50 percent means that 50 percent of the total amount of blood in the left ventricle is pushed out with each heartbeat. Ejection fraction is used as a measure of heart failure. The EF of a normal heart is typically between 50-70 percent. 41-49 percent may be considered borderline and an EF under 40 percent may be evidence of heart failure or cardiomyopathy.

The term “hazard ratio” or “HR” reflects the instantaneous probability (i.e., hazard rate) of an event (death or progression) in the experimental arm as a ratio to the probability in the comparator arm. If the HR is 1.0, there is no clear advantage for either arm. The lower the HR value, the greater the reduction in risk of death or progression for the experimental treatment arm of the study, which is calculated as 1-HR. For example, an HR of 0.84 equals a 16% relative reduction in event risk in comparison with the control arm of the study.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The term “Mayo Stage IV patients” or “Stage IV” refers to patients with stage IV disease according to the prognostic staging system established by the Mayo Clinic (Kumar et al., Revised Prognostic Staging System for Light Chain Amyloidosis Incorporating Cardiac Biomarkers and Serum Free Light Chain Measurements, J Clin Oncol 30:989-995 2012), which incorporates both cardiac biomarkers and level of amyloidogenic light chain synthesis. Collectively, patients with stage I, stage II or stage III disease are referred to herein as “Mayo Stage I-III patients” or “Stage I-III patients”. In some embodiments, a patient is identified as having Stage IV AL amyloidosis if they meet the criteria for the following three prognostic variables: troponin-T (cTnT)≥0.025 ng/mL, N-terminal pro-B-type natriuretic peptide (NT-ProBNP)≥1,800 μg/mL, and difference between involved and uninvolved light chain (FLC-diff or dFLC)≥18 mg/dL). In certain embodiments, a patient can be confirmed as Mayo Stage IV as defined by: (1) NT-proBNP≥1800 μg/mL, (2) Troponin-T>0.03 ng/mL, and (3) dFLC≥18 mg/dL.

The term “p-value” or “p” refers to a number between 0 and 1 relating to the significance of results obtained. A small p-value indicates strong evidence against the null hypothesis (i.e., the hypothesis that there is no effect), for example≤0.1, indicates statistical significance, with p≤0.001 being statistically highly significant (less than one in a thousand chance of being wrong).

The phrase “substantially from a human immunoglobulin or antibody” means that, when aligned to a human immunoglobulin or antibody amino sequence for comparison purposes, the region shares at least 80-90%, preferably 90-95%, more preferably 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably 90-95%, more preferably, 96%, 97%, 98%, or 99% identical to that of a non-human organism, e.g., a non-human mammal.

Accordingly, all regions or residues of a humanized immunoglobulin or antibody, or of a humanized immunoglobulin or antibody chain, except possibly the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

The phrases “risk reduction” and “risk of” refer to the relative risk unless specified to mean absolute risk.

### II. Methods of Treatment and Amenable Subjects

Patients amenable to treatment can be identified by determining the Mayo Stage of the patient's AL amyloidosis. Alternatively, or in addition, patients likely to receive a health benefit from the treatment can be identified by determining the 6MWT of the patient and determining the ejection fraction of the patient. Patients likely to respond positively to treatment are those with Stage IV AL amyloidosis, patients with a 6MWT of ≥150 meters and an EF of >50% at baseline, patients with Mayo Stage IV AL amyloidosis with an EF of >50% at baseline, Mayo Stage IV patients with a 6MWT of ≥150 meters and an EF of >50% at baseline, and patients with cardiac involvement . . .

Provided herein are methods of treating a human patient showing symptoms of or diagnosed with Mayo Stage IV AL amyloidosis and/or a human patient having a baseline 6MWT of ≥150 meters and a baseline EF of >50%, comprising administering to the patient a regimen of any of the antibodies or antibody formulations described herein effective to improve the health status of the patient. Some of the patients have Mayo Stage IV AL amyloidosis and a human patient having a baseline 6MWT of ≥150 meters and a baseline EF of >50%. Some patients have Mayo Stage IV AL amyloidosis and a baseline EF of >50%. Some patients have cardiac involvement. Some patients have systemic organ dysfunction attributed to AL amyloidosis, including dysfunction of the heart, kidney, liver, peripheral nervous system, gastrointestinal system, autonomic nervous system, lung, and/or soft tissue or lymphatic system.

Some methods involve determining the baseline level of troponin-T, NT-proBNP and relative levels of involved and uninvolved light chain in a patient, selecting the patient for treatment if the patient has a baseline level of cTnT≥0.025 ng/ml or >0.03 ng/ml, NT-ProBNP≥1,800 μg/mL (and <8500 μg/mL) and FLC-diff≥18 mg/dL, and administering an effective dosage of any of the antibodies disclosed herein. Some methods involve determining the 6MWT and EF of a patient at baseline and selecting the patient for treatment if the patient has a 6MWT of ≥150 meters and an EF of >50%. In some instances, Mayo

Stage IV patients with baseline 6MWT of ≥150 meters and baseline EF of >50% are selected for treatment. Some methods involve determining the Mayo Stage and EF of the patient and in some instances Mayo Stage IV patients with a baseline EF of >50% are selected for treatment. Some methods involve determining cardiac involvement of the patient and in some instances those patients are selected for treatment.

In some embodiments, the patient is treatment naïve, meaning that the patient has not previously received any treatment for AL amyloidosis. Patients amenable to treatment also include those patients who have received, are currently receiving, or will later receive an alternate therapy for treatment of AL amyloidosis or an associated condition, such as, inflammatory diseases, chronic microbial infections, malignant neoplasms, inherited inflammatory diseases, and lymphoproliferative disorders. For example, patients may also receive or have received one or more of the therapeutic agents identified herein with respect to combination therapies. As an example, patients suffering from AL amyloidosis may also receive or have received or may later receive bortezomib, melphalan, lenalidomide, prednisone, dexamethasone, cyclophosphamide, pomalidomide, carfilzomib, doxorubicin, doxycycline, daratumumab, or combinations thereof. For those patients who have previously received alternate therapies for the treatment of amyloid disease, such therapies may or may not have been successful by the relevant clinical measures, and likely did not improve health status. Additional examples of such therapies include (1) CyBorD, which is a combination therapy comprising cyclophosphamide, bortezomib and dexamethasone, (2) BMDex, which is a combination of bortezomib, melphalan and dexamethasone, (3) MDex, which is a combination of melphalan and dexamethasone, (4) LDex, which is a combination of lenalidomide and dexamethasone, (5) CLD, which is a combination of cyclophosphamide, lenalidomide and dexamethasone, (6) PomDex, which is a combination of pomalidomide and dexamethasone, and (7) CRd, which is a combination of lenalidomide, cyclophosphamide and dexamethasone. Such patients may, or may not, have experienced cardiac and/or renal improvement as a result of such treatment.

An improvement in health status can be established when the patient exhibits an improvement in the six minute walk distance (meters) outcome measure (6MWT). Also provided herein are methods of improving SF-36v2 physical component summary (PCS) score in a patient with AL amyloidosis comprising: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), and 5 to 90 minutes after completion of administering the antibody, administering a therapeutically effective amount of daratumumab to the patient, wherein the SF-36v2 score is determined at least 1 months after treatment, wherein the patient has one or more of the following: (a) Mayo Stage IV AL amyloidosis, (b) a 6 minute walk distance (6MWT)≥30 meters and ≤ 550 meters, (c) a 6MWT≥150 meters and EF>50%, (d) Mayo Stage IV and an EF>50%,

(e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥30 meters and ≤550 meters.

An improvement in the 36-Item Short Form Survey Physical Component Score (SF-36 PCS) or Short Form 36 questionnaire (including SF-36v2) can also indicate an improvement in health status of the patient. For example, a patient treated with an antibody for at least nine months who scores at least 5 points higher on the SF-36 PCS or SF-36v2 questionnaire than a different patient at the same time point who has not received the antibody has achieved an improvement in health status. In some embodiments, treatment with an antibody disclosed herein results in an increase of a patient's PCS, as measured by SF-36 or SF36v2, of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, or at least 99. In some embodiments, treatment with an antibody disclosed herein results in an increase of a patient's PCS, as measured by SF-36 or SF36v2, of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100.

Reduced length of hospitalization stays or reduced frequency of hospitalization for more than 90 days of patients treated with the antibodies disclosed herein compared to patients who have not received antibody can also indicate an improvement in health status of the patient. Improvement in health status can also be shown by longer survival of the antibody treated patient compared to untreated patients around the same time. In some embodiments, treatment with an antibody disclosed herein can reduce the risk of all-cause mortality for the treated patient by at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% relative to control treated patients, as can be determined by calculating hazard ratios between the treated patient and untreated patients.

In some embodiments, treatment with the antibodies disclosed herein can reduce the risk of all-cause mortality for the treated patient by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% relative to control treated patients, as can be determined by calculating hazard ratios between the treated patient and untreated patients. In some embodiments, treatment with the antibodies disclosed herein can reduce the risk of all-cause mortality for the treated patient by about 45%, 48.9%, 50%, 50.2%, 60%, 62.2%, 65%, 70%, 75%, 79.9%, 80% or 81.5% relative to control treated patients, as can be determined by calculating hazard ratios between the treated patient and untreated patients.

For example, the risk of all-cause mortality for some Mayo Stage IV patients can be reduced by about 50.2%, and by about 79.9% for some Mayo Stage IV patients if such patients have some level of functional reserve prior to treatment as defined by baseline 6MWT of ≥150 meters and an ejection fraction of >50%.

In some embodiments, the risk of all-cause mortality for some Mayo Stage IV patients can be reduced by at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% if they have an ejection fraction of >50%. In some embodiments, the risk of all-cause mortality for some Mayo Stage IV patients can be reduced by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% if they have an ejection fraction of >50%. In some embodiments, the risk of all-cause mortality for some Mayo Stage IV patients can be reduced by about 81.5% if they have an ejection fraction of >50%.

In some embodiments, the risk of all-cause mortality some patients having a baseline 6MWT of ≥150 meters and an ejection fraction of >50%, regardless of Mayo Stage, can be reduced by at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% if they have an ejection fraction of >50%. In some embodiments, the risk of all-cause mortality some patients having a baseline 6MWT of ≥150 meters and an ejection fraction of >50%, regardless of Mayo Stage, can be reduced by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% if they have an ejection fraction of >50%. In some embodiments, the risk of all-cause mortality in some patients having a baseline 6MWT of ≥150 meters and an ejection fraction of >50%, regardless of Mayo Stage, can be reduced by about 48.9%. The risk of cardiac mortality for some Mayo Stage IV patients can be reduced by about 62.2%.

In some embodiments, treatment with one or more antibodies disclosed herein results in an increase of a patient's 6MWT by at least 1 meter, at least 2 meters, at least 3 meters, at least 4 meters, at least 5 meters, at least 6 meters, at least 7 meters, at least 8 meters, at least 9 meters, at least 10 meters, at least 11 meters, at least 12 meters, at least 13 meters, at least 14 meters, at least 15 meters, at least 16 meters, at least 17 meters, at least 18 meters, at least 19 meters, at least 20 meters, at least 21 meters, at least 22 meters, at least 23 meters, at least 24 meters, at least 25 meters, at least 26 meters, at least 27 meters, at least 28 meters, at least 29 meters, at least 30 meters, at least 31 meters, at least 32 meters, at least 33 meters, at least 34 meters, at least 35 meters, at least 36 meters, at least 37 meters, at least 38 meters, at least 39 meters, at least 40 meters, at least 41 meters, at least 42 meters, at least 43 meters, at least 44 meters, at least 45 meters, at least 46 meters, at least 47 meters, at least 48 meters, at least 49 meters, at least 50 meters, at least 51 meters, at least 52 meters, at least 53 meters, at least 54 meters, at least 55 meters, at least 56 meters, at least 57 meters, at least 58 meters, at least 59 meters, at least 60 meters, at least 61 meters, at least 62 meters, at least 63 meters, at least 64 meters, at least 65 meters, at least 66 meters, at least 67 meters, at least 68 meters, at least 69 meters, at least 70 meters, at least 71 meters, at least 72 meters, at least 73 meters, at least 74 meters, at least 75 meters, at least 76 meters, at least 77 meters, at least 78 meters, at least 79 meters, at least 80 meters, at least 81 meters, at least 82 meters, at least 83 meters, at least 84 meters, at least 85 meters, at least 86 meters, at least 87 meters, at least 88 meters, at least 89 meters, at least 90 meters, at least 91 meters, at least 92 meters, at least 93 meters, at least 94 meters, at least 95 meters, at least 96 meters, at least 97 meters, at least 98 meters, at least 99 meters, or at least 100 meters. In some embodiments, treatment with an antibody disclosed herein results in an increase of a patient's 6MWT by about 1 meter, about 2 meters, about 3 meters, about 4 meters, about 5 meters, about 6 meters, about 7 meters, about 8 meters, about 9 meters, about 10 meters, about 11 meters, about 12 meters, about 13 meters, about 14 meters, about 15 meters, about 16 meters, about 17 meters, about 18 meters, about 19 meters, about 20 meters, about 21 meters, about 22 meters, about 23 meters, about 24 meters, about 25 meters, about 26 meters, about 27 meters, about 28 meters, about 29 meters, about 30 meters, about 31 meters, about 32 meters, about 33 meters, about 34 meters, about 35 meters, about 36 meters, about 37 meters, about 38 meters, about 39 meters, about 40 meters, about 41 meters, about 42 meters, about 43 meters, about 44 meters, about 45 meters, about 46 meters, about 47 meters, about 48 meters, about 49 meters, about 50 meters, about 51 meters, about 52 meters, about 53 meters, about 54 meters, about 55 meters, about 56 meters, about 57 meters, about 58 meters, about 59 meters, about 60 meters, about 61 meters, about 62 meters, about 63 meters, about 64 meters, about 65 meters, about 66 meters, about 67 meters, about 68 meters, about 69 meters, about 70 meters, about 71 meters, about 72 meters, about 73 meters, about 74 meters, about 75 meters, about 76 meters, about 77 meters, about 78 meters, about 79 meters, about 80 meters, about 81 meters, about 82 meters, about 83 meters, about 84 meters, about 85 meters, about 86 meters, about 87 meters, about 88 meters, about 89 meters, about 90 meters, about 91 meters, about 92 meters, about 93 meters, about 94 meters, about 95 meters, about 96 meters, about 97 meters, about 98 meters, about 99 meters, or about 100 meters.

In some embodiments, treatment with one or more antibodies herein results in an increase of a patient's 6MWT by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%. In some embodiments, treatment with one or more antibodies disclosed herein results in an increase of a patient's 6MWT by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, treatment with one or more antibodies disclosed herein results in a decrease of a patient's ejection fraction of ≤50%, ≤49%, ≤48%, ≤47%, ≤46%, ≤45%, ≤44%, ≤43%, ≤42%, ≤41%, ≤40%, ≤39%, ≤38%, ≤37%, ≤36%, ≤35%, ≤34%, ≤33%, ≤32%, ≤31%, ≤30%, ≤29%, ≤28%, ≤27%, ≤26%, ≤25%, ≤24%, ≤23%, ≤22%, ≤21%, ≤20%, ≤19%, ≤18%, ≤17%, ≤16%, ≤15%, ≤14%, ≤13%, ≤12%, ≤11%, ≤10%, ≤9%, ≤8%, ≤7%, ≤6%, ≤5%, ≤4%, ≤3%, ≤2%, ≤1%, or 0%.

In some embodiments, treatment with one or more antibodies disclosed herein results in an increase of a patient's survival by at least 1 month, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some embodiments, treatment with an antibody disclosed herein results in an increase of a patient's survival by about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months.

In some embodiments, treatment is stopped for a patient with one or more of the following:
NT-proBNP>8,500 μg/mL, or NT-proBNP is <1800 μg/mL or >8,500 μg/mL
Troponin-T≤0.03 ng/ml or <0.025 ng/ml
dFLC<18 mg/dL,
Absolute neutrophil count (ANC)<1.0×10⁹/L
Platelet count<75×10⁹/L
Hemoglobin<9 g/dL
Total bilirubin>2 times the upper limit of normal (× ULN)
Aspartate aminotransferase (AST)/serum glutamic oxaloacetic transaminase (SGOT)>3× ULN
Alanine aminotransferase (ALT)/serum glutamic pyruvic transaminase (SGPT)>3× ULN
Alkaline phosphatase (ALP)>5×ULN (except for subjects with hepatomegaly and isozymes specific to liver, rather than bone)
Estimated glomerular filtration rate (eGFR)<30 mL/min/1.73 m² as estimated by the Chronic Kidney Disease Epidemiology Collaboration (CKD-EPI) equation
Seated systolic blood pressure<90 or >180 mmHg
Distance walked during a 6MWT is <30 meters or >550 meters
Undergoes ASCT or organ transplant
Myocardial infarction, uncontrolled angina, severe uncontrolled ventricular arrhythmias, or electrocardiographic (ECG) evidence of acute ischemia
Severe valvular stenosis (e.g. aortic or mitral stenosis with a valve area<1.0 cm²) or severe congenital heart disease
ECG evidence of acute ischemia or active conduction system abnormalities with the exception of any of the following: First degree AV-block, Second degree AV-block Type 1 (Mobitz Type 1/Wenckebach type), Right or left bundle branch block, and Atrial fibrillation with a controlled ventricular rate (uncontrolled [>110 bpm] ventricular rate is not allowed [determined by an average of three beats in Lead II or three representative beats if Lead II is not representative of the overall EKG])
Peripheral neuropathy assessed as National Cancer Institute-Common Terminology Criteria for Adverse Events (NCI-CTCAE) Grade 2 with pain, Grade 3, or Grade 4
Active malignancy with the exception of any of the following: adequately treated basal cell carcinoma, squamous cell carcinoma, or in situ cervical cancer, adequately treated Stage I cancer from which the subject is currently in remission and has been in remission (e.g. for 2 years), low-risk prostate cancer with Gleason score<7 and prostate-specific antigen<10 mg/mL, and any other cancer from which the subject has been disease-free (e.g. for ≥2 years)
Epilepsy or seizure disorder with the exception of childhood febrile seizures Suitable antibodies, formulations and treatment regimens for the methods and uses d is closed herein are discussed in greater detail below.

### III. Antibodies

The methods of the disclosure include administering to a patient one or more antibodies. For example, the methods of the disclosure include administering to a patient an antibody that specifically bind to immunoglobulin light chain. Examples include antibodies that compete with 11-1F4 for binding to immunoglobulin light chain, and antibodies that compete with 2A4 or 7D8 for binding to human amyloid A peptide, or specifically bind to the same epitope as 11-1F4 (U.S. Pat. No. 8,105,594), 2A4 or 7D8 (U.S. Pat. Nos. 7,928,203). In some embodiments, the antibody is a humanized version of 2A4. In some embodiments, the antibody is a chimeric or humanized version of 11-1F4, such as, for example, Ch mAb 11-1F4, CAEL-101. In some embodiments, the antibody is one that is disclosed in U.S. Patent Application Publication Number 20190038745A1, U.S. Patent Application Publication Number 20200002410A1, and U.S. Pat. No. 10,046,050. In some embodiments, the antibody comprises a light chain variable region comprising three complementarity determining regions set forth as SEQ ID NOs: 3, 4 and 5, and a heavy chain variable region comprising three complementarity determining regions set forth as SEQ ID NOs: 6, 7 and 8. In some embodiments, the light chain variable region comprises the amino acid sequence set forth as SEQ ID NO: 1. In some embodiments, the heavy chain variable region comprises the amino acid sequence set forth as SEQ ID NO: 2.

In other methods, the antibody comprises light chain and heavy chain variable regions of a murine, chimeric, or humanized 2A4 antibody, or of a murine, chimeric, or humanized 7D8 antibody, as described in U.S. Pat. No. 7,928,203 and PCT International Publication No. WO 2009/086539, each of which is incorporated herein by reference in its entirety, and the light chain and heavy chain variable region sequences described in the referenced patent and publication are specifically incorporated by reference herein. Some formulations for the methods disclosed herein are described in U.S. Pat. No. 9,089,529 and PCT International Publication No. WO 2013/063284.

In some embodiments, the antibody comprises a light chain comprising an amino acid sequence set forth as SEQ ID NO: 10 and a heavy chain comprising an amino acid sequence set forth as any one of SEQ ID NOs: 11-13. For example, the antibody can comprise a light chain comprising an amino acid sequence set forth as SEQ ID NO: 10 and a heavy chain comprising an amino acid sequence set forth as SEQ ID NO: 12. The antibody can include, or not include, the leader sequences of the above-noted light chain and heavy chain amino acid sequences. In some embodiments, the antibody is birtamimab (CAS Registry No. 1608108-91-3).

In other methods, the antibody is a fragment of a 2A4 or 7D8 antibody, including chimeric and humanized versions thereof, such as a Fab fragment, a Fab′ fragment, a F(ab′) 2 fragment, F (ab) c, Dab, nanobody or Fv. As discussed in greater detail below, the antibodies can be administered as a pharmaceutical formulation.

In some embodiments, the methods of the disclosure include administering to a patient a second antibody. In some embodiments, the second antibody is daratumumab.

Daratumumab is a targeted therapy (IgG1k human monoclonal antibody) that targets CD38. CD38 is a cell surface glycoprotein which is highly expressed on myeloma cells. It is expressed at low levels on normal myeloid and lymphoid (normal) type of white blood cells. When daratumumab binds to CD38, it inhibits the growth of CD38 expressing cells (e.g., cancer cells and plasma cells) and induces apoptosis (cell death) directly through mediated cross linking and by immune mediated tumor cell lysis through complement dependent cytotoxicity, antibody cell mediated cytotoxicity, and antibody dependent cellular phagocytosis.

DARZALEX® (daratumumab) is approved by the FDA. See DARZALEX® [Package Insert] Janssen Biotech (2015) https://www.accessdata.fda.gov/scripts/cder/daf/index.cfm?event-overview.process& ApplNo=761036. It is available as an injection, for intravenous use. DARZALEX is a CD38-directed cytolytic antibody indicated for the treatment of adult patients with multiple myeloma. Recommended dose is 16 mg/kg actual body weight. See Package Insert for full description of indications and usage and dosage and administration.

DARZALEX FASPRO® (daratumumab and hyaluronidase-fihj) is approved by the FDA. See DARZALEX FASPRO® [Package Insert] Janssen Biotech (2020) https://www.accessdata.fda.gov/scripts/cder/daf/index.cfm?event-overview.process&ApplNo=761145. It is available as injection, for subcutaneous use. DARZALEX FASPRO is a combination of daratumumab, a CD38-directed cytolytic antibody, and hyaluronidase, an endoglycosidase, indicated for the treatment of adult patients with:
multiple myeloma, and
light chain (AL) amyloidosis in combination with bortezomib, cyclophosphamide and dexamethasone in newly diagnosed patients. This indication is approved under accelerated approval based on response rate. Continued approval for this indication may be contingent upon verification and description of clinical benefit in a confirmatory trial(s).

DARZALEX FASPRO is not indicated and is not recommended for the treatment of patients with light chain (AL) amyloidosis who have NYHA Class IIIB or Class IV cardiac disease or Mayo Stage IIIB outside of controlled clinical trials. See Package Insert for full description of indications and usage and dosage and administration.

The DARZALEX FASPRO package insert states “Serious or fatal cardiac adverse reactions occurred in patients with light chain (AL) amyloidosis who received DARZALEX FASPRO in combination with bortezomib, cyclophosphamide and dexamethasone [see Adverse Reactions (6.1)]. Serious cardiac disorders occurred in 16% and fatal cardiac disorders occurred in 10% of patients. Patients with NYHA Class IIIA or Mayo Stage IIIA disease may be at greater risk. Patients with NYHA Class IIIB or IV disease were not studied.”

No clear survival benefit has been observed for patients with AL amyloidosis who are administered daratumumab. Kastritis et al. reported in 2021 that at median follow-up time of 11.4 months in the Phase 3 ANDROMEDA study 56 patients died-27 in the daratumumab group (daratumumab+CyBorD) and 29 in the control group (CyBorD). Kastritis, et al. N Engl J Med. 2021 Jul. 1;385 (1): 46-58.

Additionally, in ANDOMEDA early deaths (i.e. <60 days from start of therapy) occurred in 13 patients in each group, and overall death was considered as related adverse events in 23 patients (11.9%) in the daratumumab-CyBorD and in 14 patients (7.4%) in CyBorD group; disease progression as a cause of death was reported in 1.0% with daratumumab-CyBorD versus 4.8% with CyBorD. As expected, most adverse events that were considered as leading to death, as well as mortality overall, were mostly observed in patients with advanced cardiac amyloidosis and all patients who died due to cardiac complications had amyloidotic heart involvement. Theodorakakou, et al., Ther Adv Hematol. 2021 Nov. 23; 12:1-10.

### IV. Pharmaceutical Formulations and Products

In some embodiments disclosed herein, the antibodies of the present disclosure can be administered to a patient as a pharmaceutical formulation, for example, comprising in addition to the antibodies, a histidine buffer, trehalose, and polysorbate 20. In some such formulations used in the methods described above, the antibodies are present at a concentration within the range from about 1 mg/mL to about 100 mg/mL; the histidine buffer is present at a concentration within the range from about 20 mM to about 30 mM; the trehalose is present at a concentration within the range from about 210 mM to about 250 mM; the polysorbate 20 present at a concentration within the range from about 0.005% to about 0.05% by weight; and the pH is within the range from about 6 to about 7. Some suitable formulations for the methods disclosed herein are described in greater detail below.

In some formulations, the antibodies are present at a concentration within the range from about 5 mg/mL to about 100 mg/mL. In some formulations, the antibodies are present at a concentration within the range from about 5 mg/mL to about 15 mg/mL. In some formulations, the antibodies are present at a concentration within the range from about 25 mg/mL to about 75 mg/mL. For example, the antibodies may be present at a concentration of about 10 mg/mL, or present at a concentration of about 50 mg/mL. The antibodies may be present in a sterile liquid dosage form of about 50 mg/vial to about 500 mg/vial, or greater. For example, the antibodies may be present in a sterile liquid dosage form of about 100 mg/vial. In another, non-limiting example, the antibodies may be present as a sterile, lyophilized dosage form that may be reconstituted with sterile liquid dosage form of about 500 mg/vial. In another, non-limiting example, the antibodies may be present as a sterile, lyophilized dosage form that may be reconstituted with sterile liquid of about 10 mL for a dosage form of about 50 mg/mL or about 500 mg/vial.

Antibodies used in the disclosed formulations can be coupled with a therapeutic moiety, such as a cytotoxic agent, a radiotherapeutic agent, an immunomodulator, a second antibody (e.g., to form an antibody heteroconjugate), or any other biologically active agent that facilitates or enhances the activity of a chimeric or humanized 2A4 or a chimeric or humanized 7D8 antibody or daratumumab. Representative therapeutic moieties include agent known to be useful for treatment, management, or amelioration of amyloid disease or symptoms of amyloid disease.

Therapeutic moieties and/or detectable substances may be coupled or conjugated directly to a murine, chimeric or humanized 2A4 antibody or a murine, chimeric or humanized 7D8 antibody or daratumumab, or indirectly, through an intermediate (e.g., a linker) using techniques known in the art. See e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., Immunol. Rev., 1982, 62:119-58. Antibodies used in the disclosed formulations also include modified forms of murine, chimeric or humanized 2A4 antibodies, or murine, chimeric or humanized 7D8 antibodies, or daratumumab which have increased in vivo half-lives relative to the corresponding unmodified antibodies. Such modified forms may be prepared, for example, by glycosylation, acetylation, pegylation, phosphorylation, amidation, derivatization by known protecting/blocking groups, proteolytic cleavage, linkage to a cellular ligand or other protein, etc. As one example, representative methods for antibody half-life extension are described in PCT International Publication No. WO 02/060919.

The histidine buffer may be present in some formulations at a concentration of about 25 mM. In some formulations, the histidine buffer comprises L-histidine and L-histidine HCl monohydrate. For example, in some formulations, L-histidine is present at a concentration within the range from about 16 mM to about 22 mM and L-histidine HCl monohydrate is present at a concentration within the range from about 4 mM to about 8 mM.

In some formulations, trehalose is present at a concentration from about 210 mM to about 250 mM, for example, about 230 mM. In some formulations, a different non-reducing sugar is used, such as sucrose, mannitol, or sorbitol.

In some formulations, polysorbate 20 is present at a concentration within the range of about from about 0.005% to about 0.05% by weight, for example, 0.005%, 0.01%, 0.015%, 0.02%, 0.025%, 0.03%, 0.035%, 0.04%, 0.045%, or 0.05%. Alternatively, in some formulations, polysorbate 20 is present at a concentration within the range of about from about 0.05 g/L, 0.1 g/L, 0.15 g/L, 0.2 g/L, 0.25 g/L, 0.3 g/L, 0.35 g/L, 0.4 g/L, 0.45 g/L, or 0.5 g/L. Some formulations include polysorbate 20 at a concentration of 0.2 g/L.

Some formulations are characterized by a pH within the range of about 6-7, for example, a pH of 6.0, 6.1, 6.2, 6.3, 6.4, 6.5, 6.6, 6.7, 6.8, 6.9, or 7.0. Some formulations have a pH of about 6.5. Some formulations are characterized by an osmolality of about 300 mOsm/kg. A bulking agent may also be included some formulations.

Typically, the formulations are sterile, for example, as accomplished by sterile filtration using a 0.2 μm or a 0.22 μm filter. The formulations disclosed herein are also generally stable upon freezing and thawing.

Optionally, formulations disclosed herein may further comprise other excipients, such as saccharides, polyols, and amino acids (e.g., arginine, lysine, and methionine).

The present disclosure also provides formulations substantially free of surfactant, inorganic salts, additional sugars, and/or other excipients, i.e., less than about less than 0.0005%, less than 0.0003%, or less than 0.0001% of such compounds.

An exemplary formulation comprises an antibody comprising a light chain comprising an amino acid sequence set forth as SEQ ID NO: 10 and a heavy chain comprising an amino acid sequence set forth as any one of SEQ ID NOs: 11, 12, or 13, which is present at a concentration of about 50 mg/mL, a histidine buffer present at a concentration of about 25 mM, trehalose present at a concentration of about 230 mM, polysorbate 20 present at a concentration of about 0.2 g/L, and a pH of about 6.5. Some formulations comprise an antibody comprising a light chain comprising an amino acid sequence set forth as SEQ ID NO: 10 and a heavy chain comprising an amino acid sequence set forth as SEQ ID NO: 12, which is present at a concentration of about 50 mg/mL, a histidine buffer present at a concentration of about 25 mM, trehalose present at a concentration of about 230 mM, polysorbate 20 present at a concentration of about 0.2 g/L, and a pH of about 6.5. Some formulations comprise birtamimab, which is present at a concentration of about 50 mg/mL, a histidine buffer present at a concentration of about 25 mM, trehalose present at a concentration of about 230 mM, polysorbate 20 present at a concentration of about 0.2 g/L, and a pH of about 6.5.

The methods disclosed herein involve pharmaceutical products comprising lyophilized antibody drug substance and instructions for reconstitution and use. For example, a representative pharmaceutical product can comprise: (a) a vial comprising about 100 mg antibody in powder form; (b) instructions for reconstitution of the antibody; and (c) instructions for preparing the reconstituted antibody for infusion, wherein (i) the antibody comprises a light chain comprising an amino acid sequence set forth as SEQ ID NO: 10 and a heavy chain comprising an amino acid sequence set forth as any one of SEQ ID NOs: 12-15; and (ii) the reconstitution instructions require reconstitution with water for injection to an extractable volume of 10 mL.

### V. Treatment Regimens

As used herein, the terms “treat” and “treatment” refer to the alleviation or amelioration of one or more symptoms or effects associated with the disease, prevention, inhibition or delay of the onset of one or more symptoms or effects of the disease, lessening of the severity or frequency of one or more symptoms or effects of the disease, and/or increasing or trending toward desired outcomes as described herein.

Desired outcomes of the treatments disclosed herein vary according to the amyloid disease and patient profile and are readily determinable to those skilled in the art. Desired outcomes include an improvement in the patient's health status. Generally, desired outcomes include measurable indices such as reduction and/or clearance of pathologic amyloid fibrils, decreased or inhibited amyloid aggregation and/or deposition of amyloid fibrils, and increased immune response to pathologic and/or aggregated amyloid fibrils. Desired outcomes also include amelioration of amyloid disease-specific symptoms. For example, desired outcomes for the treatment of AL amyloidosis include a decrease in the incidence or severity of known symptoms, including organ dysfunction, peripheral and autonomic neuropathy, carpal tunnel syndrome, macroglossia, restrictive cardiomyopathy, arthropathy of large joints, immune dyscrasias, myelomas, as well as occult dyscrasias.

For example, the 6-minute walk test (6MWT) can be a surrogate endpoint used to assess cardiac functional response (Pulido et al., The six-minute walk test in patients with AL amyloidosis: a single centre case series, British Journal of Haematology, 2017, 177, 388-394). It measures the distance patients can walk in 6 minutes along thirty meter long hallways. For example, the mean 6-minute walk distance (6MWT) of AL amyloidosis patients with cardiac involvement has been shown to be significantly shorter than the distance walked by AL amyloidosis patients without cardiac involvement. Further, increased distance walked is correlated with a decrease in mortality. In some embodiments, a baseline of a 6MWT>300 meters is independent of Mayo staging, including stage IIIb in predicting survival. In some embodiments, a≥300 meter 6MWT is a meaningful threshold for a patient treated for at least 12 months. In some embodiments, a 33 meter improvement in a 6MWT is a clinically meaningful value in cardiopulmonary disorders after treatment of at least 12 months.

Provided herein are methods of treating a patient having AL amyloidosis, including: administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient; and 5 to 90 minutes after completion of administering the antibody, administering a therapeutically effective dosage of daratumumab to the patient, where the patient has Mayo Stage IV AL amyloidosis.

Also provided herein are methods of treating a patient having AL amyloidosis, including: (A) determining one or more of the following: the Mayo Stage of the patient's AL amyloidosis; the 6 minute walk test distance (6MWT) of the patient; the ejection fraction (EF) of the patient; cardiac involvement of the patient; (B) selecting the patient for treatment with (a) an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), and (b) daratumumab, if the patient has Mayo Stage IV AL amyloidosis; (C) administering an effective dosage of the antibody to the selected patient; and (D) 5 to 90 minutes after completion of administering the antibody, administering a therapeutically effective dosage of daratumumab to the selected patient.

Also provided herein are methods of treating a patient having AL amyloidosis, including:
(a) determining one or more of the following:
   i. the Mayo Stage of the patient's AL amyloidosis;
   ii. the 6 minute walk test distance (6MWT) of the patient;
   iii. the ejection fraction (EF) of the patient;
   iv. cardiac involvement of the patient;
(b) selecting the patient for treatment with (i) an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), and (ii) daratumumab, if the patient:
   i. has Mayo Stage IV AL amyloidosis; or
   ii. has a 6MWT≥30 meters and ≤550 meters; or
   iii. has a 6MWT≥150 meters and an EF>50% at baseline; or
   iv. has Mayo Stage IV and a 6MWT≥30 meters and ≤550 meters; or
   v. has Mayo Stage IV and EF>50% at baseline; or
   vi. has Mayo Stage IV, a 6MWT≥150 meters and an EF>50% at baseline;
   vii. has cardiac involvement;
   viii. has Mayo Stage IV AL amyloidosis and has cardiac involvement; or
   ix. has Mayo Stage IV AL amyloidosis, cardiac involvement, and a 6MWT≥30 meters and ≤550 meters
b. administering an effective dosage of the antibody to the selected patient; and
c. 5 to 90 minutes after completion of administering the antibody, administering a therapeutically effective dosage of daratumumab to the selected patient.

In some embodiments, the patient has a 6MWT≥30 meters and ≤550 meters.

Also provided herein are methods of treating a patient having AL amyloidosis, comprising: administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient; and 5 to 90 minutes after completion of administering the antibody, administering a therapeutically effective dosage of daratumumab to the patient, wherein the patient has one or more of the following: (a) Mayo Stage IV AL amyloidosis, (b) a 6 minute walk distance (6MWT)≥30 meters and ≤550 meters, (c) a 6MWT≥150 meters and EF>50%, (d) Mayo Stage IV and an EF>50%, (e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥ 30 meters and ≤550 meters.

Also provided herein are methods of treating a patient having Mayo Stage IV AL amyloidosis and having NYHA class III or NYHA class IV, the method comprising: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient's NYHA class is reduced by at least two classes. In some embodiments, the patient's NYHA class is assessed nine or more months after treatment.

In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Also provided herein are methods of treating a patient with AL amyloidosis, comprising: determining that the patient has a 6 minute walk distance (6MWT)≥30 meters and ≤550 meters or a 6MWT≥150 meters and/or an ejection fraction (EF)>50%; selecting the patient for treatment with an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), and daratumumab; administering an effective dosage of the antibody to the selected patient; and 5 to 90 minutes after completion of administering the antibody, administering a therapeutically effective amount of daratumumab to the selected patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

Also provided herein are methods of treating a patient with AL amyloidosis who has a demonstrated 6 minute walk distance (6MWT) of ≥30 meters and ≤550 meters or a 6MWT greater than or equal to 150 meters and/or an ejection fraction (EF) of more than 50%, comprising: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105; and 5 to 90 minutes after completion of administering the antibody, administering a therapeutically effective amount of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Also provided herein are methods of reducing mortality or the risk of mortality in a patient having Mayo Stage IV AL amyloidosis and having NYHA class III or NYHA class IV, the method comprising: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of after administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

In some embodiments, a second antibody (e.g., daratumumab) is administered to the patient between about 5 minutes to about 120 minutes after completion of administering the first antibody. In some embodiments, a second antibody (e.g., daratumumab) is administered about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, or about 120 minutes after completion of administering the first antibody. In some embodiments, the second antibody (e.g., daratumumab) is administered at the same time as the first antibody.

Also provided herein are methods of reducing the risk of mortality in a patient with AL amyloidosis by at least 45%, comprising: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), 6MWT and 5 to 90 minutes after completion of administering the antibody, administering a therapeutically effective amount of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

A positive change in health-related quality of life is also a desired outcome of the disclosed therapies, including, for example, as measured by the SF-36 and or SF-36v2 Health Survey (White et al., Psychometric validation of the SF-36 Health Survey in light chain amyloidosis: results from community-based and clinic-based samples, Patient Related Outcome Measures 2017:8 157-167). The SF-36v2 involves scores that represent eight dimensions of function and well-being: physical functioning, role limitations due to physical problems, bodily pain, general health perceptions, vitality, social functioning, role limitations due to emotional problems and mental health, and summary scores, such as physical component summary (PCS) and mental component summary (MCS). Higher SF-36 scores represent better health. Desired outcomes of the disclosed therapies are generally quantifiable measures as compared to a control or baseline measurement. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same amyloid disease as the individual being treated, who is about the same age as the individual being treated (to ensure that the stages of the disease in the treated individual and the control individual are comparable), but who has not received treatment using the disclosed antibody formulations. In this case, efficacy of the disclosed antibody formulations is assessed by a shift or trend away from measurable indices in the untreated control. Alternatively, a control individual is a healthy individual, who is about the same age as the individual being treated. In this case, efficacy of the disclosed antibody formulations is assessed by a shift or trend toward from measurable indices in the healthy control. Changes or improvements in response to therapy are generally statistically significant and described by a p-value less than or equal to 0.1, less than 0.05, less than 0.01, less than 0.005, or less than 0.001 may be regarded as significant.

Mortality is the primary cause of disease-related death in late stage Mayo patients. As such, mortality (e.g., all causes or cardiovascular related) can be used as a valid clinical endpoint and can also be used to asses cardiac functional response. In some embodiments, cardiac mortality is less than or equal to about 12 months for late stage Mayo amyloidosis. In some embodiments, cardiac mortality is about 1 year to about 4 years for intermediate stage Mayo amyloidosis. In some embodiments, cardiac mortality is about 4 years or greater for early stage Mayo amyloidosis. In some embodiments, treatment of a patient results in at least a 15% relative reduction rate in the risk of all-cause mortality. In some embodiments, treatment can include a 15%, 16%, 17%, 18%, 19%, 20%, 21%, 22%, 23%, 24%, or a 25% relative reduction rate in the risk of all-cause mortality as compared to the risk of mortality in a control population. In some embodiments, treatment can include at least a 25%, a 30%, a 35%, a 40%, a 45%, a 50%, a 55%, a 60%, a 65%, a 70%, a 75%, an 80%, an 85%, a 90%, a 95%, or a 100% relative reduction in the risk all-cause mortality as compared to the risk of mortality in a control population. In some embodiments, treatment of a patient results in at least a 15% relative reduction rate in the risk of cardiac mortality. In some embodiments, treatment can include a 15%, 16%, 17%, 18%, 19%, 20%, 21%, 22%, 23%, 24%, or a 25% relative reduction rate in the risk cardiac mortality as compared to the risk of mortality in a control population. IN some embodiments, treatment can include at least a 25%, a 30%, a 35%, a 40%, a 45%, a 50%, a 55%, a 60%, a 65%, a 70%, a 75%, an 80%, an 85%, a 90%, a 95%, or a 100% relative reduction in the risk of cardiac mortality as compared to the risk of mortality in a control population.

In some embodiments, a relative reduction in mortality or the risk of mortality (e.g., all-cause mortality, cardiac mortality) is the risk of mortality within 3 months, 6 months, 9 months, 12 months, 18 months, 24 months, 36 months, 48 months, 60 months, or 72 months. Thus, provided herein are methods of reducing the risk of mortality in a patient having Mayo Stage IV AL amyloidosis and having NYHA class III or NYHA class IV, the method comprising administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount, and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments the risk of mortality is assessed nine or more months after treatment.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Hospitalization is also recognized as a highly relevant, valid clinical endpoint of morbidity in heart failure. In some embodiments, a relative reduction in hospitalization is clinically meaningful. In some embodiments, a 20% relative reduction in hospitalizations is clinically meaningful as compared to the risk of hospitalization in a control population. In some embodiments, treatment results in a 5%, a 10%, a 15%, 25%, a 30%, a 35%, a 40%, a 45%, a 50%, a 55%, a 60%, a 65%, a 70%, a 75%, an 80%, an 85%, a 90%, a 95%, or a 100% relative reduction in hospitalizations as compared to the risk of hospitalization in a control population. In some embodiments, the average duration of life gained outside of the hospital is a patient center goal (e.g., days alive out of the hospital (DAOH) or days at home). In some embodiments, an increase in the number of days alive out of the hospital is clinically meaningful. In some embodiments, the number of DAOH increases by at least 20 days after 1 month of treatment. In some embodiments, the number of DAOH increases by at least 100 days after six months of treatment. In some embodiments, the number of DAOH increases by at least 300 after twelve months of treatment. In some embodiments, the number of DAOH increases by at least 600 after 24 months of treatment. In some embodiments, the number of DAOH increases by at least 900 after 48 months of treatment as compared DAOH in a control population. In some embodiments, the number of DAOH increases by 100, 200, 300, 400, 500, 600, 700, 800, 900, 1000, 1100, 1200, 1300, 1400, or 1500 after treatment as compared to DAOH in a control population.

Thus, provided herein are methods of reducing the risk of hospitalization in a patient having Mayo Stage IV AL amyloidosis, the method comprising: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV

AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Also provided herein are methods of increasing the number of days alive out of hospital (DAOH) in a patient Mayo Stage IV amyloidosis, the method comprising: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Biomarkers are also recognized as a method to clinically assess response to treatment and guide treatment management. In some embodiments, the biomarker is NTproBNP. In some embodiments, NTproBNP is a cardiac biomarker. In some embodiments, NTproBNP is a valid clinical endpoint. For example, a response to treatment with birtamimab that results in a>30% and >300 ng/L decline from a baseline of >650 ng/L is a meaningful response. In some embodiments, the response to treatment is assessed by NTproBNP biomarker is a graded criteria. In some embodiments, a≤30% reduction in NTproBNP from a baseline is considered no response. In some embodiments, a 31% to 60% reduction in NTproBNP from a baseline is considered a partial response. In some embodiments, a>60% reduction in NTproBNP from a baseline to a nadir NTproBNP>400 μg/mL is considered a very good partial response. In some embodiments, a nadir NTproBNP<400 μg/mL is considered a complete response.

Thus, provided herein are methods of reducing NTproBNP level in a patient having Mayo Stage IV AL amyloidosis, the method comprising: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

In some embodiments, NTproBNP is reduced by at least 31% from a baseline after 3 months of treatment. In some embodiments, NTproBNP is reduced by at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59% from a baseline (e.g., after treatment for 3, 6, 9, 12, 15, 18, 21, 24, 27, 30, 33, or 36 months). In some embodiments, NTproBNP is reduced by at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% from a baseline (e.g., after treatment for 3, 6, 9, 12, 15, 18, 21, 24, 27, 30, 33, or 36 months). In some embodiments, NTproBNP is reduced by at least 60% from a baseline to a nadir>400 μg/ml after 3 months of treatment. In some embodiments, NTproBNP is reduced to a nadir<400 μg/ml after 3 months of treatment. In some embodiments, NTproBNP is reduced by at least 45% from a baseline after 6 months of treatment. In some embodiments, NTproBNP is reduced by at least 60% from a baseline after 6 months of treatment. In some embodiments, NTproBNP is reduced by at least 60% from a baseline to a nadir>400 μg/ml after 6 months of treatment. In some embodiments, NTproBNP is reduced to a nadir<400 μg/ml after 3 months of treatment.

Also provided herein are methods of improving cardiac response rate in a patient having Mayo Stage IV AL amyloidosis, the method comprising: administering a therapeutically effective amount of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

In some embodiments, the cardiac response rate increases at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% as compared to a baseline. In some embodiments, the cardiac response rate increases at least 30% after 6 months of treatment. In some embodiments, the cardiac response rate increases at least 50% after 12 months of treatment. In some embodiments, the cardiac response rate increases at least 75% after 12 months of treatment.

Also provided herein are methods of improving a six minute walk test (6MWT) in a patient having Mayo Stage IV AL amyloidosis, the method comprising administering a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

In some embodiments, the 6MWT is at least 300 meters after treatment for 12 months. In some embodiments, the 6MWT improves at least 33 meters after 12 months of treatment.

In some embodiments the 6MWT achieved is at least 300 meters after treatment for 18 months. In some embodiments, the 6MWT improves at least 33 meters after 18 months of treatment as compared to baseline.

The Kansas City Cardiomyopathy Questionnaire (KCCQ) score is a recognized measure of quality of life (QOL) in heart failure. In some embodiments, the KCCQ score is used as a valid clinical endpoint. In some embodiments, a 5 point change in KCCQ score is a clinically minimal important difference. In some embodiments, a 5 point change in KCCQ score is a small change in KCCQ score. In some embodiments, a 10 point change KCCQ score is a moderate to large change in KCCQ score. In some embodiments, a 20 point change in KCCQ score is a large to very large change in KCCQ score. In some embodiments, the KCCQ score increases by at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, or at least 90 compared to a baseline. In some embodiments, the increase in KCCQ score after 3, 6, 9, 12, 15, 18, 21, 24, 27, 30, 33, or 36 months of treatment.

In some embodiments, the KCCQ score increases by at least 5 after 3 months of treatment as compared to baseline. In some embodiments, the KCCQ score increases by at least 10 after 3 months of treatment as compared to baseline. In some embodiments, the KCCQ score increased by at least 15 after 3 months of treatment as compared to baseline. In some embodiments, the KCCQ score improves by at least 20 after 3 months of treatment as compared to baseline. In some embodiments, the KCCQ score improves by at least 5 after 12 months of treatment as compared to baseline. In some embodiments, the KCCQ score improves by at least 10 after 12 months of treatment as compared to baseline. In some embodiments, the KCCQ score improves by at least 15 after 12 months of treatment as compared to baseline. In some embodiments, the KCCQ score improves by at least 20 after 12 months of treatment as compared to baseline.

Also provided herein are methods of increasing a Kansas City Cardiomyopathy Questionnaire (KCCQ) score in a patient having Mayo Stage IV AL amyloidosis, the method comprising: administering a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

Major Organ Deterioration Progression Free Survival (MOD-PFS) is a composite endpoint defined as any one of the following events (e.g., whichever event comes first): death, cardiac deterioration (requiring a cardiac transplant, left ventricular assist device or intra-aortic balloon pump), end stage renal disease requiring hemodialysis or rent transplant, or hematological progression per consensus guidelines. In some embodiments, progression of MOD-PFS is measured by >30% and >300 ng/L increase in NTproBNP. In some embodiments, the response to treatment is assessed by NTproBNP biomarker is a graded criteria. In some embodiments, a≤30% reduction in NTproBNP from a baseline is considered no response. In some embodiments, a 31% to 60% reduction in NTproBNP from a baseline is considered a partial response. In some embodiments, a>60% reduction in NTproBNP from a baseline to a nadir>400 μg/mL is considered a very good partial response. In some embodiments, a nadir NTproBNP<400 μg/mL is considered a complete response.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Thus provided herein are methods of increasing Major Organ Deterioration Progression Free Survival in a patient having Mayo Class IV AL amyloidosis, the method comprising: administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and 5 to 90 minutes after completion of administering the antibody, administering a therapeutically effective dosage of daratumumab to the patient, wherein the patient has one or more of the following: (a) a 6 minute walk distance (6MWT)≥30 meters and ≤550 meters, (b) a 6MWT≥150 meters and EF>50%, (c) Mayo Stage IV and an EF>50%, (d) Mayo Stage IV and a 6MWT≥30 meters and ≤550 meters, (e) Mayo Stage IV and a 6MWT≥150 meters and EF>50%, (f) has cardiac involvement; (g) has Mayo Stage IV AL amyloidosis and has cardiac involvement; or (h) has Mayo Stage IV AL amyloidosis, cardiac involvement, and a 6MWT≥30 meters and ≤550 meters.

In some embodiments, NTproBNP is reduced by at least 31% from a baseline after 3 months of treatment. In some embodiments, NTproBNP is reduced by at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59% from a baseline (e.g., after treatment for 3, 6, 9, 12, 15, 18, 21, 24, 27, 30, 33, or 36 months). In some embodiments, NTproBNP is reduced by at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% from a baseline (e.g., after treatment for 3, 6, 9, 12, 15, 18, 21, 24, 27, 30, 33, or 36 months). In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

In some embodiments, the NTproBNP level is reduced by at least 31% from a baseline after 3 months of treatment. In some embodiments, the NTproBNP level is reduced by at least 45% from a baseline after 3 months of treatment. In some embodiments, the NTproBNP level is reduced by at least 60% from a baseline after 3 months of treatment. In some embodiments, the NTproBNP level is reduced by at least 60% from a baseline to a nadir>400 μg/ml after 3 months of treatment. In some embodiments, NTproBNP is reduced to a nadir<400 μg/ml after 3 months of treatment. In some embodiments, NTproBNP is reduced by at least 45% from a baseline after 6 months of treatment. In some embodiments, NTproBNP is reduced by at least 60% from a baseline after 6 months of treatment. In some embodiments, NTproBNP is reduced by at least 60% from a baseline to a nadir>400 μg/ml after 6 months of treatment. In some embodiments, NTproBNP is reduced to a nadir<400 μg/ml after 3 months of treatment.

Treatment typically entails multiple dosages over a period of time. Treatment can be monitored by assaying antibody, or employing radiolabeled SAP Scintigraphy over time. If the response falls, a booster dosage may be indicated. Changes in the health status of the patients can be monitored based on outcome measures such as 6MWT, SF-36 PCS (SF-36v2), hospitalizations and survival as discussed in greater detail above. In addition, the response of patients with AL amyloidosis to treatment can be monitored by assessing cardiac markers, such as NT-proBNP and/or troponin-T, serum creatine, and/or alkaline phosphatase; by performing serum free light chain (SFLC) assays, quantitative immunoglobulin assays, biopsies, serum protein electrophoresis (SPEP), urine protein electrophoresis (UPEP), serum, urine immunofixation electrophoresis (IFE), and/or organ imaging techniques. An exemplary complete response (CR) can be determined from response criteria including negative IFE of serum and urine, normal K/\ ration and/or <5% plasma cells in bone marrow. An exemplary very good partial response (VGPR) can be determined from a dFLC of <40 mg/L. An exemplary partial response (PR) can be determined from a dFLC decrease of ≥50%. In the kidney, a response to treatment can be determined, for example, from a≥50% reduction (e.g., >0.5 g/24 hours) in 24 hour urine protein excretion in the absence of either a reduction in eGFR of >25% or an increase in serum creatine of ≥0.5 mg/dL. In the liver, a response to treatment can be determined, for example, from a≥50% reduction in initially elevated alkaline phosphatase or a>2 cm reduction in liver size on CT scan or MRI. In the heart, a response to treatment can be determined, for example, from a>30% and >300 ng/L reduction in NT-proBNP in patients with baseline of NT-proBNP of >650 ng/L. In the kidney, a response to treatment can be determined, for example, from a>30% decrease in proteinuria or a decrease in proteinuria to <0.5 g/24 hours in the absence of renal progression. Neuropathy responders are generally characterized by <2 point increase in NIS-LL from baseline. Improvement in neuropathy (e.g., improved nerve function) is determined from a decrease in the NIS-LL from baseline. Improvement in health status can also be determined from a decrease in the frequency of hospitalizations, a decrease in hospitalizations of greater than ninety days, or from longer survival relative to an untreated different patient with a similar prognosis upon diagnosis, for example, AL amyloidosis patients with cardiac involvement.

Also provided herein are methods of reducing global longitudinal strain (GLS) in a patient with AL amyloidosis comprising: administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), 6MWT and 5 to 90 minutes after completion of administering the antibody, administering a therapeutically effective amount of daratumumab to the patient, wherein the patient has one or more of the following: (a) Mayo Stage IV AL amyloidosis, (b) a 6 minute walk distance (6MWT)≥30 meters and ≤550 meters, (c) a 6MWT≥150 meters and EF>50%, (d) Mayo Stage IV and an EF>50%, (e) Mayo Stage IV and a 6MWT≥30 meters and ≤550 meters, (f) Mayo Stage IV and a 6MWT≥150 meters and EF>50%, (g) has cardiac involvement; (h) has Mayo Stage IV AL amyloidosis and has cardiac involvement; or (i) has Mayo Stage IV AL amyloidosis, cardiac involvement, and a 6MWT≥30 meters and ≤550 meters.

In some embodiments, the patient has Mayo Stage IV AL amyloidosis. In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the selected patient has cardiac involvement. In some embodiments, the selected patient has Mayo Stage IV AL amyloidosis and has cardiac involvement. In some embodiments, the selected patient has Mayo Stage IV AL amyloidosis, cardiac involvement, and a 6MWT≥30 meters and ≤550 meters.

Also provided herein are methods of reducing global longitudinal strain (GLS) in a patient having Mayo Stage IV AL amyloidosis, including administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient.

In some embodiments, the administering results in a reduction of GLS of about 0.1% to about 10% in the patient. In some embodiments, the administering results in a reduction of GLS of about 0.5% to about 10% in the human subject. In some embodiments, the administering results in a reduction of GLS of about 1% to about 10% in the human subject. In some embodiments, the administering results in a reduction of GLS of at least about 0.5% in the human subject. In some embodiments, the administering results in a reduction of GLS of at least about 1.0% in the human subject. In some embodiments, the administering results in a reduction of GLS of at least about 2.0% in the human subject. In some embodiments, the administering results in a reduction of GLS of at least about 10% in the human subject.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement.

In some embodiments, the patient has Mayo Stage IV AL amyloidosis, cardiac involvement, and a 6MWT≥30 meters and ≤550 meters.

Also provided herein are methods of reducing global longitudinal strain (GLS) in a patient having Mayo Stage IV AL amyloidosis, including: administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient; and 5 to 90 minutes after completion of administering the antibody, administering a therapeutically effective dosage of daratumumab to the patient.

In some embodiments, the administering results in a reduction of GLS of about 0.1% to about 10% in the patient. In some embodiments, the administering results in a reduction of GLS of about 0.5% to about 10% in the human subject. In some embodiments, the administering results in a reduction of GLS of about 1% to about 10% in the human subject.

In some embodiments, the administering results in a reduction of GLS of at least about 0.5% in the human subject. In some embodiments, the administering results in a reduction of GLS of at least about 1.0% in the human subject. In some embodiments, the administering results in a reduction of GLS of at least about 2.0% in the human subject. In some embodiments, the administering results in a reduction of GLS of at least about 10% in the human subject.

In some embodiments, the patient has a 6MWT≥150 meters and an EF>50%. In some embodiments, the patient has a 6MWT≥30 meters and ≤550 meters. In some embodiments, the patient has an EF>50%. In some embodiments, the patient has a 6MWT≥150 meters. In some embodiments, the patient has cardiac involvement. In some embodiments, the patient has Mayo Stage IV AL amyloidosis and has cardiac involvement.

In some embodiments, the patient has Mayo Stage IV AL amyloidosis, cardiac involvement, and a 6MWT≥30 meters and ≤550 meters.

In some embodiments, the administering of the antibody or the antigen-binding fragment thereof results in an improved cardiac systolic function as measured by global longitudinal strain (GLS) of about 0.1% to about 99% (e.g., about 0.1% to about 99%, about 0.1% to about 95%, about 0.1% to about 90%, about 0.1% to about 85%, about 0.1% to about 80%, about 0.1% to about 75%, about 0.1% to about 70%, about 0.1% to about 65%, about 0.1% to about 60%, about 0.1% to about 55%, about 0.1% to about 50%, about 0.1% to about 45%, about 0.1% to about 40%, about 0.1% to about 35%, about 0.1% to about 30%, about 0.1% to about 25%, about 0.1% to about 20%, about 0.1% to about 15%, about 0.1% to about 10%, about 0.1% to about 5%, about 0.1% to about 1%, about 1% to about 99%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 99%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to the GLS in the human subject prior to the administering (e.g., when administered using any of the exemplary dosages and/or frequencies described herein).

In some embodiments, the administering of the antibody or the antigen-binding fragment thereof results in an improved cardiac systolic function as measured by global longitudinal strain (GLS) of at least about at least about 0.5%, at least about 1%, at least about 1.5%, at least about 2%, at least about 2.5%, at least about 3%, at least about 3.5%, at least about 4%, at least about 4.5%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%) (e.g., when administered using any of the exemplary dosages and/or frequencies described herein).

In some embodiments, the administering of the antibody or the antigen-binding fragment thereof results in an improved cardiac systolic function as measured by GLS of at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.1%, at least about 3.2%, at least about 3.3%, at least about 3.4%, at least about 3.5%, at least about 3.6%, at least about 3.7%, at least about 3.8%, at least about 3.9%, at least about 4%, at least about 4.1%, at least about 4.2%, at least about 4.3%, at least about 4.4%, at least about 4.5%, at least about 4.6%, at least about 4.7%, at least about 4.8%, at least about 4.9%, or at least about 5%) (e.g., when administered using any of the exemplary dosages and/or frequencies described herein).

The antibody formulation can be administered intravenously or subcutaneously in dosage ranges from about 0.5 mg/kg to about 30 mg/kg of the host body weight. For example, dosages can be about 0.5 mg/kg body weight, about 1.0 mg/kg, about 1.5 mg/kg, about 2.0 mg/kg, about 4.0 mg/kg, about 5.0 mg/kg, about 8.0 mg/kg, about 10 mg/kg, about 15 mg/kg, about 16 mg/kg, about 20 mg/kg, about 24 mg/kg, about 25 mg/kg, or about 30 mg/kg body weight. The dosages can also be administered according to body surface area from about 0.5 mg/m² to about 500 mg/m², for example, 0.5, 5, 10, 50, 100, 250 or 500 mg/m². For intravenous dosing, an amount of the antibody formulation sufficient to achieve the desired dosage for the individual patient is transferred from one or more vials to one or more intravenous bags containing a liquid (e.g., saline) and administered to the patient.

Antibody is usually administered on multiple occasions. An exemplary treatment regimen entails administration once per every two weeks, once a month, or once every 3 to 6 months. For example, patients can receive the antibody formulation once every four weeks as a cycle, for example every twenty-eight days. The dosing frequency can be adjusted depending on the pharmacokinetic profile of the antibody formulation in the patient. For example, the half-life of the antibody may warrant a two week frequency of dosing. In some embodiments, the pharmaceutical formulation is administered intravenously every 28 days with an antibody dosage of about 24 mg/kg. For example, some patients may receive an intravenous dose of about 24 mg/kg birtamimab every 28 days. For example, some patients may receive an intravenous dose of about 24 mg/kg birtamimab every 28 days (+5 days). In certain embodiments, a minimum of 21 days is required between doses. For some such patients, the birtamimab formulation transferred to the intravenous bag was first reconstituted from a lyophilized formulation to a formulation having a pH of about 6.5 and comprising about 50 mg/ml birtamimab, about 25 mM histidine buffer, about 230 mM trehalose and about 0.2 g/L polysorbate 20. For some patients the desired dosage can be administered subcutaneously without dilution from a vial containing any of the formulations disclosed herein.

In some embodiments disclosed herein, the antibodies are administered to the patient for at least 8 months, at least 9 months, at least 10 months, at least 11 months, at least 12 months, at least 13 months, at least 14 months, at least 15 months, at least 16 months, at least 18 months, at least 19 months, at least 20 months, at least 21 months, at least 22 months, at least 23 months, at least 24 months, or for a longer period of time. For example, the pharmaceutical formulation is administered to the patient for a duration effective to achieve or maintain an improvement in health status as indicated by an increase in 6MWT or SF-36v2

PCS score, or long enough to achieve or maintain a lower risk of mortality relative to an untreated patient. For some patients, the lower risk can be established after at least 8 months of treatment. For some patients, the lower risk can be established after at least 9 months of treatment. For some patients, the lower risk can be established after at least 12 months of treatment or after at least 18 months of treatment or after twenty-four months of treatment.

For these patients, a lower risk of mortality correlates with longer survival times relative to untreated patients.

Also disclosed herein are combination therapies for treatment or prophylaxis of AL amyloidosis. Such combination therapies are performed by administering an antibody formulation disclosed herein in conjunction with one or more second therapeutic agents, such as another therapy to treat or effect prophylaxis of AL amyloidosis. Combination therapies as disclosed herein may also be performed in conjunction with a second therapy is used to treat or effect prophylaxis of a disease or condition associated with amyloid disease, such as an inflammatory disease, a chronic microbial infection, a neoplasm (including malignant neoplasms), an inherited inflammatory disease, and/or a lymphoproliferative disorder.

Numerous treatments are available in commercial use, in clinical evaluation, and in pre-clinical development, any of which could be selected for use in combination with the disclosed antibody formulations. Such treatments can be one or more compounds or treatments selected from, but not limited to several major categories, namely, (i) non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate); (ii) steroids (e.g., cortisone, dexamethasone, hydrocortisone, methylprednisolone, prednisolone, prednisone, triamcinolone); (iii) DMARDs, i.e., disease modifying antirheumatic drugs (e.g., cyclosporine, azathioprine, methotrexate, leflunomide, cyclophosphamide, hydroxychloroquine, sulfasalazine, D-penicillamine, minocycline, and gold); (iv) recombinant proteins (e.g., ENBREL® (etanercept, a soluble TNF receptor) and REMICADE® (infliximab) a chimeric monoclonal anti-TNF antibody); (v) stem cell transplantation; and/or (vi) chemotherapy. Patients with AL amyloidosis may also receive treatment regimens that include drugs or combinations of drugs often used to treat hematological malignancies, such as melphalan, prednisone, dexamethasone, lenalidomide (REVLIMID®), proteosome inhibitors such as bortezomib (VELCADE®) and carfilzomib (KYPROLIS®), and CD38 agents at dosages in the range of the standard of care.

When performing a combination therapy, the two or more drug substances are administered simultaneously or sequentially in any order, i.e., a formulation disclosed herein is administered prior to administering a second drug substance, concurrently with a second drug substance, or subsequent to administration of a second drug substance. For example, a combination therapy may be performed by administering a first therapy prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) administering a second agent/therapy such as daratumumab.

The dosage, frequency and mode of administration of each component of the combination can be controlled independently. For example, one therapeutic agent/therapy may be administered orally three times per day, while the second therapeutic agent/therapy may be administered intramuscularly once per day. Combination therapy may be given in on-and-off cycles that include rest periods. The compounds may also be admixed or otherwise formulated together such that one administration delivers both compounds. In this case, each therapeutic agent is generally present in an amount of 1-95% by weight of the total weight of the composition. Alternatively, an antibody formulation disclosed herein and a second therapeutic agent can be formulated separately and in individual dosage amounts.

Drug combinations for treatment can be provided as components of a pharmaceutical pack.

Preferably, the disclosed combination therapies elicit a synergistic therapeutic effect, i.e., an effect greater than the sum of their individual effects or therapeutic outcomes. Measurable therapeutic outcomes are described herein. For example, a synergistic therapeutic effect may be an effect of at least about two-fold greater than sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

Some methods of the disclosure include treating a subject having AL amyloidosis by determining one or more of the following prognostic indicators: (1) the Mayo Stage of the patient's AL amyloidosis, (2) the 6 minute walk distance (6MWT) and ejection fraction (EF) of the patient, and/or the Mayo Stage and the EF of the patient. Once the prognostic indicator(s) has been determined, a patient is selected the patient for treatment if the patient meets one of the following treatment criteria: (1) has Mayo Stage IV AL amyloidosis; (2) has a 6MWT≥30 meters and ≤550 meters; (3) has a 6MWT≥150 meters and an EF>50% at baseline; (4) has Mayo Stage IV and a 6MWT≥30 meters and ≤550 meters; (5) has Mayo Stage IV and EF>50% at baseline; (6) has Mayo Stage IV, a 6MWT≥150 meters and an EF>50% at baseline; (7) has cardiac involvement; (8) has Mayo Stage IV AL amyloidosis and has cardiac involvement; or (9) has Mayo Stage IV AL amyloidosis, cardiac involvement, and a 6MWT≥30 meters and ≤550 meters. Treatment includes administering an effective dosage of one or more antibodies disclosed herein.

In one method of the disclosure, a patient meeting or more of the prognostic indicators is treated with birtamimab (24 mg/kg) supplied as a sterile, lyophilized dosage form in a 20/25 mL vial containing 500 mg birtamimab. Each vial may be reconstituted with 9.6 mL sterile water for injection (WFI) to a concentration of 50 mg/mL resulting in a buffered, isotonic, preservative-free solution. Birtamimab is administered once every 28 days as an initial 120 (+10)-minute IV infusion. If the subject tolerates the initial infusion, subsequent infusions may be administered over 60 (+10) minutes. Dose are administered at intervals of at least 21 days.

Patients may also be treated with concomitant standard of care chemotherapy, which may include, for example, bortezomib administered subcutaneously on a weekly basis. In some embodiments, concomitant treatment with the following is not allowed: other investigational agents (e.g., drugs not approved for any indication), myeloablative chemotherapy with ASCT, organ transplant, and doxycycline. In some embodiments, steroids are not allowed prior to study entry for the treatment of AL amyloidosis. In some embodiments, initiation of daratumumab treatment at any time other than at randomization is prohibited.

### EXAMPLES

The following examples have been included to illustrate modes disclosed herein. Certain aspects of the following examples are described in terms of techniques and procedures found or contemplated by the present co-inventors to work well in the practice disclosed herein. In light of the present disclosure and the general level of skill in the art, those of skill appreciate that the following examples are intended to be exemplary only and that numerous changes, modifications, and alterations may be employed without departing from the scope of the disclosure.

Example 1-a Phase 3, Randomized, Multicenter, Double-Blind, Placebo-Controlled, Efficacy and Safety Study of Birtamimab Plus Standard of Care Vs. Placebo Plus Standard of Care in Mayo Stage Iv Subjects with Light Chain (Al) Amyloidosis

The primary objective of the study (NEOD001-301) is to evaluate the efficacy of birtamimab plus standard of care when administered intravenously in Mayo Stage IV subjects with AL amyloidosis by assessing time to all-cause mortality. Secondary objectives are to evaluate birtamimab plus standard of care on the following: (1) change from baseline to month 9 in the 6 Minute Walk Test (6MWT) distance (meters) and (2) change from baseline to month 9 in health related quality of life using the Short Form 36 questionnaire (SF-36v2).

Newly diagnosed Mayo Stage IV subjects with AL amyloidosis receive birtamimab plus local standard of care chemotherapy. The initial first-line chemotherapy regimen must include bortezomib. Subjects will be stratified at randomization based on their 6MWT distance (<300 meters vs.≥300 meters) and initiation of daratumumab treatment at randomization (yes vs. no).

Subjects will remain on study until study completion, which will occur when approximately 47 primary endpoint events (all-cause mortality) have been reached. The primary endpoint is time to all-cause mortality. The distribution of survival times will be compared between treatment groups using a log rank test.

An interim analysis will be conducted when approximately 50% of the events (24) have occurred. The O'Brien-Fleming group sequential methodology will be used to divide the overall study significance level between the interim (p=0.0108) and the final analysis (p=0.0984). If overwhelming efficacy (p<0.0108) is achieved at the interim analysis, the study will be stopped early.

If the subject discontinues study drug prior to the end of the study, but is willing to continue to participate in study visits, the subject should have an Early Treatment Discontinuation (ETD) visit within 28-35 days after the last study drug administration and then have assessments every third month (see Table 2). All visits after the ETD Visit should occur on schedule, that is, at the time when the visit would have occurred had the subject remained on study drug.

Subject screening will occur during the 28 days prior to the first administration of study drug on Month 1-Day 1. The screening period may be extended upon approval by the Medical Monitor. Screening assessments are listed in Table 1, herein.

Two screening 6MWTs are required before the first administration of study drug. The first screening 6MWT is required to be performed between Days-28 and 5, at least 4 days prior to the second Screening 6MWT, which should be performed within 3 days prior to Month 1 Day 1 (i.e., on Day 3 to Day 1). The post baseline 6MWTs may be performed on the same day as study drug administration and must be completed prior to study drug infusion. If all eligibility requirements are met, Month 1-Day 1 assessments are completed, and treatment is initiated. Each visit is denoted by its “month” and “day” such that the first study drug infusion day is denoted as Month 1-Day 1; subsequent months use sequential numbers (e.g., the second dose is administered on Month 2-Day 1). “Cycle” is reserved to denote administration of chemotherapy. Assessment and visit windows are described in the Schedule of Events (Table 1). All doses of study drug will be administered at the study site, starting on the day of randomization and then every 28 days (±2 days for months 1 through 3 and ±5 days for all subsequent months) from the previous Month X-Day 1 visit until the EOT/ETD Visit. For Months 1 through 3, subjects are assessed weekly, although not all visits are required to be at the study site. For Month 3 and all subsequent months until the end of the study, subjects are only required to return to the study site every 28 days for Day 1 dosing of study drug.

First-line chemotherapy must be a bortezomib-containing regimen, with bortezomib administered subcutaneously (SC), weekly. The first administration of chemotherapy, including bortezomib and daratumumab for those subjects stratified to initiate daratumumab at randomization, will be administered after Month 1 Day 1 study drug administration (following the post-study drug infusion observation period) such that Month 1-Day 1 of the study is equivalent to Cycle 1 Day 1 of chemotherapy. In addition to the visits outlined above, during the first cycle of chemotherapy, the subject must return to the study site for each weekly administration of bortezomib and for assessments prior to the administrations. During the second and third cycles of chemotherapy, bortezomib must be administered at the study site during the Month 2-Day 1, Month 2-Day 15, and Month 3-Day 1 visits (i.e., Cycle 2 Day 1, Cycle 2-Day 15, and Cycle 3-Day 1, respectively). If, for any reason in the opinion of the Investigator, the subject should continue to be seen weekly at the study site (e.g., toxicity that appears to exceed the anticipated side effects of the chemotherapy), then the other Cycle 2 and Cycle 3 weekly bortezomib administrations may be performed at the study site, as well. At the Investigator's discretion, if the subject is not experiencing any unanticipated or significant toxicity, the subject may be administered the Cycle 2-Days 8 and 22 and the Cycle 3 Days 8, 15 and 22 bortezomib by their local physician, rather than by the Investigator. Within 1 day prior to or on the day of each administration of bortezomib by the local physician, a healthcare professional must obtain pre-dose vital signs and central laboratory samples. However, if bortezomib is administered on a Monday (or there is an intervening holiday), then it is acceptable for the Homecare visit to take place on the previous Friday.

In the event that bortezomib doses are missed, the chemotherapy cycles may become misaligned with the monthly study drug dosing. In this case, the weekly visits during Months 1 through 3 should continue as described above in order to closely monitor subjects' health during the initial months of concomitant chemotherapy. Throughout the study, monthly doses of study drug should not be delayed or skipped due to adjustments that are made to chemotherapy dosing.

Safety and efficacy assessments are performed at each visit.

Autologous stem cell transplant is not allowed while on study.

### Inclusion Criteria (Subjects Must Meet all of the Following Criteria):

1. Aged≥18 years and legal age of consent according to local regulations.
2 Newly diagnosed and AL amyloidosis treatment naïve.
3. Bone marrow demonstrating clonal plasma cells.
4. Confirmed diagnosis of AL amyloidosis by the following:
   Histochemical diagnosis of amyloidosis determined by polarizing light microscopy of green birefringent material in Congo red-stained tissue specimens OR characteristic electron microscopy appearance, AND.
   Confirmatory immunohistochemistry OR mass spectroscopy of AL amyloidosis.
5. If the subject meets any of the following:
   Is black or of African descent,
   Is over 75 years of age with concurrent monoclonal gammopathy.,
   Has a history of familial amyloidosis and has concurrent monoclonal gammopathy,
AND

No tissue is available for typing, and the subject has echocardiographic evidence of amyloidosis and biopsy-proven amyloidosis with a monoclonal gammopathy, then the subject must have gene sequencing consistent with transthyretin (TTR) wild type (i.e., no TTR mutation present) AND must score 0 in technetium-99m-3,3-diphosphono-1,2 propanodicarboxylic acid (^{99m}Tc-DPD; Rapezzi 2011), hydroxymethylenediphosphonate (^{99m}Tc-HMDP; Galat 2015), or pyrophosphate (^{99m}Tc-PYP; Bokhari 2013) scintigraphy.
6. Cardiac involvement as defined by all of the following:
   Past documented or presently noted clinical signs and symptoms supportive of a diagnosis of heart failure in the setting of a confirmed diagnosis of AL amyloidosis in the absence of an alternative explanation for heart failure.
   Either an endomyocardial biopsy demonstrating AL amyloidosis or an echocardiogram demonstrating a mean left ventricular wall thickness at diastole>12 mm in the absence of other causes (e.g., severe hypertension, aortic stenosis), which would adequately explain the degree of wall thickening,
7. Confirmed Mayo Stage IV as defined by:
   NT-proBNP≥1800 μg/mL, and
   Troponin-T≥0.025 ng/ml (mcg/L) or high sensitivity cardiac troponin T≥40 ng/L, and
   dFLC≥18 mg/dL.
8. Planned first-line chemotherapy contains bortezomib administered subcutaneously (SC) weekly.
9. Adequate bone marrow reserve, hepatic function, and renal function, as demonstrated by:
   Absolute neutrophil count (ANC)≥1.0×109/L,
   Platelet count≥75×109/L,
   Hemoglobin≥9 g/dL,
   Total bilirubin≤2 times the upper limit of normal (× ULN), (except for subjects with Gilbert's syndrome, in which case direct bilirubin≤x ULN)
   Aspartate aminotransferase (AST)/serum glutamic oxaloacetic transaminase (SGOT)≤3×ULN,
   Alanine aminotransferase (ALT)/serum glutamic pyruvic transaminase (SGPT)≤3×ULN,
   Alkaline phosphatase (ALP)≤5×ULN (except for subjects with hepatomegaly and isozymes specific to liver, rather than bone),
   Estimated glomerular filtration rate (eGFR)≥30 mL/min/1.73 m² as estimated by the Chronic Kidney Disease Epidemiology Collaboration (CKD-EPI) equation.
10. Seated systolic blood pressure 90-180 mmHg.
11. Distance walked during each Screening 6MWT is ≥30 meters and ≤550 meters.
12. Women of childbearing potential (WOCBP) must have two negative pregnancy tests during Screening, the second within 24 hours prior to the first administration of study drug, and must agree to use highly effective physician-approved contraception from Screening to 90 days following the last study drug administration.
13. Male subjects must be surgically sterile or must agree to use a barrier method, together with the use of highly effective physician-approved contraception by their female partner of childbearing potential, from Screening to 90 days following the last study drug administration.
14. Ability to understand and willingness to sign an informed consent form prior to initiation of any study procedures.

### Exclusion Criteria (Subjects Must Meet None of the Following Criteria):

1. Non-AL amyloidosis.
2. NT-proBNP>8,500 μg/mL.
3. Meets the International Myeloma Working Group (IMWG) definition of Multiple Myeloma, except for malignancy biomarker of involved/uninvolved serum free light chain ratio≥100.
4. Subject is eligible for and plans to undergo ASCT or organ transplant during the study.
5. Symptomatic orthostatic hypotension that in the medical judgment of the Investigator would interfere with subject's ability to safely receive treatment or complete study assessments.
6. Myocardial infarction, uncontrolled angina, severe uncontrolled ventricular arrhythmias, or electrocardiographic (ECG) evidence of acute ischemia, within 6 months prior to the Month 1-Day 1 Visit.
7. Severe valvular stenosis (e.g. aortic or mitral stenosis with a valve area<1.0 cm²) or severe congenital heart disease.
8. ECG evidence of acute ischemia or active conduction system abnormalities with the exception of any of the following:
   First degree AV-block,
   Second degree AV-block Type 1 (Mobitz Type 1/Wenckebach type),
   Right or left bundle branch block,
   Atrial fibrillation with a controlled ventricular rate (uncontrolled [>110 bpm] ventricular rate is not allowed [determined by an average of three beats in Lead II or three representative beats if Lead II is not representative of the overall EKG]).
9. Peripheral neuropathy assessed as National Cancer Institute-Common Terminology Criteria for Adverse Events (NCI-CTCAE) Grade 2 with pain, Grade 3, or Grade 4.
10. Subject is receiving oral or IV antibiotics, antifungals or antivirals within 1 week of Month 1-Day 1 with the exception of prophylactic oral agents.
11. Prior treatment with hematopoietic growth factors, transfusions of blood or blood products within 1 week of Month 1-Day 1.
12. Prior radiotherapy within 4 weeks of Month 1-Day 1.
13. Major surgery within 4 weeks of Month 1-Day 1 or planned major surgery during the study.
14. Active malignancy with the exception of any of the following:
   Adequately treated cutaneous basal cell carcinoma, squamous cell carcinoma, or in situ cervical cancer,
   Adequately treated Stage I cancer from which the subject is currently in remission and has been in remission for 2 years,
   Low-risk prostate cancer with Gleason score<7, prostate-specific antigen<10 mg/mL, and a stage of cancer at most cT2a, cNO, and CM0.
   Any other cancer from which the subject has been disease-free for ≥2 years.
15. History of severe allergy to any of the components of birtamimab such as histidine/L histidine hydrochloride monohydrate, trehalose dehydrate, or polysorbate 20 or history of Grade≥3 infusion-related AEs or hypersensitivity to another monoclonal antibody, or known hypersensitivity to diphenhydramine (or an equivalent H1 antihistamine) or acetaminophen (or its equivalent, paracetamol).
16. Known, unresolved, or active HIV, hepatitis B, hepatitis C, or SARS-COV-2 infection.
17. Prior treatment with plasma cell-directed chemotherapy, birtamimab, daratumumab, 11-1F4, anti-serum amyloid P antibody, doxycycline for amyloid, or other investigational treatment directed at amyloid.
18. Treatment with another investigational agent within 30 days of Month 1-Day 1.
19. Women who are pregnant or lactating.
20. Any condition which could interfere with, or the treatment for which might interfere with, the conduct of the study or which would, in the opinion of the Investigator, unacceptably increase the subject's risk by participating in the study.
21 Subject is under legal custodianship.
22 History of epilepsy or seizure disorder with the exception of childhood febrile seizures.
23. Waldenström's macroglobulinemia and/or immunoglobulin M (IgM) monoclonal gammopathy.

### Dosage, Administration, Schedule

Study Drug: Study drug consists of intravenous birtamimab or placebo. The birtamimab dose is 24 mg/kg; however, the maximum dose administered is not to exceed 2500 mg. Therefore, subjects with a weight of 104.2 kg or greater will receive the maximum dose of 2500 mg.

Birtamimab (24 mg/kg) is supplied as a sterile, lyophilized dosage form in a 20/25 mL vial containing 500 mg birtamimab. Each vial is reconstituted with 9.6 mL sterile water for injection (WFI) to a concentration of 50 mg/mL resulting in a buffered, isotonic, preservative-free solution. Study drug is administered once every 28 days as an initial 120 (±10)-minute IV infusion. If the subject tolerates the initial infusion, subsequent infusions may be administered over 60 (±10) minutes. The length of the infusion may be extended over a longer period of time if and when it is clinically indicated. A minimum of 21 days between doses is required.

Premedication: All subjects are premedicated for each dose of study drug with 25 mg diphenhydramine (or an equivalent dose of a H1 antihistamine) and 650 mg acetaminophen (or an equivalent paracetamol dose in accordance with local practice) within 30-90 minutes prior to study drug administration.

Standard of Care Chemotherapy: All subjects receive concomitant standard of care chemotherapy, which must include bortezomib administered subcutaneously on a weekly basis for the initial, first-line chemotherapy regimen. Subsequent chemotherapy regimens may be prescribed as per standard of care at the Investigator's discretion. Bortezomib should be administered according to the approved prescribing information and local institutional practices. The initial first-line chemotherapy regimen may also include daratumumab. The initiation of daratumumab treatment at randomization is allowed at the discretion of the Investigator; initiation at any other time during the study is prohibited. When chemotherapy, which may include daratumumab, is administered on same day as study drug, the chemotherapy must be administered after completion of administration of study drug.

Antiviral prophylaxis is required.

### Efficacy Endpoints

Primary Efficacy Endpoint: Time to all-cause mortality.

Secondary Efficacy Endpoints: Change from baseline to Month 9 in the 6MWT distance (meters); Change from baseline to Month 9 in the Physical Component Summary (PCS) score of the SF-36v2.

### Statistical Considerations

Analysis Populations. The Intent to Treat (ITT) Population and the Safety Population will include all randomized subjects with Mayo Stage IV AL amyloidosis who receive any amount of study drug (birtamimab or placebo). The ITT Population will be the primary population used for efficacy analyses. The Safety Population will be the primary population used for safety analyses.

### Efficacy Analyses.

Interim Analysis—An interim analysis will be conducted when approximately 50% (or 24) of the events have occurred. Using the O'Brien-Fleming group sequential methodology, the overall significance level of 0.10 will be divided between the interim analysis (p<0.0108) and final analysis (p<0.0984). Results of the interim analysis will be evaluated to determine if the trial can be stopped early for overwhelming efficacy.

Primary Analysis—The primary endpoint is time to all-cause mortality. For all-cause mortality, all deaths occurring after the first infusion of study drug (i.e., Study Day 1) through the study's last subject last visit will be included.

The distribution of the time to all-cause mortality will be summarized using the Kaplan-Meier method. Birtamimab will be compared to placebo using the log rank test, stratified by initiation of daratumumab treatment at randomization. A sensitivity analysis using the unstratified log-rank test may also be conducted.

The hazard ratio, 90% CI, and 95% CI will be determined based on the Cox regression model stratified by the initiation of daratumumab use at randomization.

Using a log-rank test, birtamimab and the placebo control will be compared at a significance level of 0.0984 at the final analysis.

Secondary Efficacy Analyses—The following secondary endpoints will be evaluated:
Change from baseline to Month 9 in the 6MWT distance (meters).
Change from baseline to Month 9 in the PCS score of the SF-36v2

The 6MWT distance (meters) change from baseline at Month 9 will be analyzed using a restricted maximu likelihood (REML) based mixed-effect model for repeated measures (MMRM) model including fixed effects for randomization stratification (<300 meters vs.≥300 meters) and treatment group, categorical time point, and the treatment group x time point interaction, and with the baseline value included as a covariate.

The SF-36v2 PCS score change from baseline at Month 9 will be analyzed using aREML based MMRM model including fixed effects for treatment group, categorical time point, and the treatment group x time point interaction, and with the baseline value included as a covariate. The unstructured covariance model will be used.

TABLE 1
Schedule of Events

| | | | M1 | | M2 | | M3 | | M4 |
|---|---|---|---|---|---|---|---|---|---|
| | SCR¹ | | | D8, | | [symbol] 8², | | D8², | D1 |
| | D-28 | D-3 [symbol] | | 15, | | 15 [symbol] | | 15², | (±5) |
| | to | to | | 22 | D1 | [symbol] 2² | D1 | 22² | Study drug will be administered |
| Assessments | D- [symbol] | D-1 [symbol] | D1 [symbol] | (±2) | (±2 [symbol] | (±2 [symbol] | (±2) | (±2) | monthly until the EOT/ETD Visit |
| Written Informed Consent | X | | | | | | | | |
| Eligibility Review | X | | | | | | | | |
| Medical History⁴ | X | X | | | | | | | |
| Confirmation of AL Amyloidosis⁵ | X | | | | | | | | |
| SF-36v2 Health Survey⁶ | | X | | | X | | X | | |
| Echocardiogram | X⁷ | | | | | | | | |
| 12-lead Triplicate ECG⁸ | X | | X | | X | | X | | X |
| Complete PE⁹ | X | | | | | | | | |
| Symptom-Directed PE¹⁰ | | | X | | X | | X | | X |
| NYHA Class | X | | X | | X | | X | | X |
| SARS-CoV-2 Test¹¹ | X | X | | | | | | | |
| Local Laboratory | | | | | | | | | |
| Hematology & Chemistry¹² | | | | X | X | X¹³ | X | X¹³ | X |
| Serum/Urine Pregnancy (WOCBP) | | | X¹⁴ | | X¹⁵ | | X¹⁵ | | X¹⁵ |
| Central Laboratory¹⁶ | | | | | | | | | |
| Hematology & Chemistry | X | X | | X | X | X | X | X | X |
| Amylase | X | X | | | X | | X | | X |
| Coagulation¹⁷ | | X | | | X | | X | | X |
| Complements C₃, C₄ | | X | | | | | X | | |
| Troponin-T | X | X | | | X | | X | | X |
| NT-proBNP¹⁸ | X | X | | | X | | X | | X |
| Tryptase | X | | | | | | | | |
| sFLCs | X | X | | | X | | X | | X |
| Serum Pregnancy (WOCBP) | X | | | | | | | | |
| Urinalysis | X | X | | | X | | X | | X |
| 6MWT¹⁹ | X²⁰ | X²⁰ | | | | | X | | |
| Bioanalytical Laboratory | | | | | | | | | |
| Anti-birtamimab Antibody (predose) | | | X | | X | | X | | X |
| Randomization²¹ | | | X | | | | | | |
| Premedication Administration²² | | | X | | X | | X | | X |
| Vital Signs²³ | X | | X | X | X | X | X | X | X |
| Study Drug Infusion²⁴ | | | X | | X | | X | | X |
| Chemotherapy & Antiviral Prophylaxis²⁵ | | | X²⁶ | X²⁶ | X²⁶ | X^{26, 27} | X²⁶ | X²⁷ | X²⁶ |
| Adverse Event Assessment | | | X | X | X | X¹² | X | X¹² | X |
| Concomitant Medications | X | X | X | X | X | X¹² | X | X¹² | X |
| Vital Status Assessment | | | | | | | | | |

| | | | | | | | | | Monthly | |
|---|---|---|---|---|---|---|---|---|---|---|
| | M5 | M6 | M7 | M8 | M9 | M10 | M11 | M12 | after | |
| | D1 | D1 | D1 | D1 | D1 | D1 | D1 | D1 | M12 | |
| | (±5) | (±5) | (±5) | (±5) | (±5) | (±5) | (±5) | (±5) | D1 (±5) | |
| | Study drug will be administered | | | | | | | | | EOT/ |
| Assessments | monthly until the EOT/ETD Visit | | | | | | | | | ETD³ |
| Written Informed Consent | | | | | | | | | | |
| Eligibility Review | | | | | | | | | | |
| Medical History⁴ | | | | | | | | | | |
| Confirmation of AL Amyloidosis⁵ | | | | | | | | | | |
| SF-36v2 Health Survey⁶ | | X | | | X | | | | | X |
| Echocardiogram | | X⁷ | | | | | | X⁷ | Every 6^{th} mo⁷ | X⁷ |
| 12-lead Triplicate ECG⁸ | X | X | X | X | X | X | X | X | X | X |
| Complete PE⁹ | | | | | | | | | | X |
| Symptom-Directed PE¹⁰ | X | X | X | X | X | X | X | X | X | |
| NYHA Class | X | X | X | X | X | X | X | X | X | X |
| SARS-CoV-2 Test¹¹ | | | | | | | | | | |
| Local Laboratory | | | | | | | | | | |
| Hematology & Chemistry¹² | X | X | X | X | X | X | X | X | X | |
| Serum/Urine Pregnancy (WOCBP) | X¹⁵ | X¹⁵ | X¹⁵ | X¹⁵ | X¹⁵ | X¹⁵ | X¹⁵ | X¹⁵ | X¹⁵ | |
| Central Laboratory¹⁶ | | | | | | | | | | |
| Hematology & Chemistry | X | X | X | X | X | X | X | X | X | X |
| Amylase | X | X | X | X | X | X | X | X | X | X |
| Coagulation¹⁷ | X | X | X | X | X | X | X | X | X | X |
| Complements C₃, C₄ | | X | | | X | | | X | Every 3rd mo | X |
| Troponin-T | X | X | X | X | X | X | X | X | X | X |
| NT-proBNP¹⁸ | X | X | X | X | X | X | X | X | X | X |
| Tryptase | | | | | | | | | | |
| sFLCs | X | X | X | X | X | X | X | X | X | X |
| Serum Pregnancy (WOCBP) | | | | | | | | | | X |
| Urinalysis | X | X | X | X | X | X | X | X | X | X |
| 6MWT¹⁹ | | X | | | X | | | | | X |
| Bioanalytical Laboratory | | | | | | | | | | |
| Anti-birtamimab Antibody (predose) | X | X | X | X | X | X | X | X | X | X |
| Randomization²¹ | | | | | | | | | | |
| Premedication Administration²² | X | X | X | X | X | X | X | X | X | |
| Vital Signs²³ | X | X | X | X | X | X | X | X | X | X |
| Study Drug Infusion²⁴ | X | X | X | X | X | X | X | X | X | |
| Chemotherapy & Antiviral Prophylaxis²⁵ | X²⁶ | X²⁶ | X²⁶ | X²⁶ | X²⁶ | X²⁶ | X²⁶ | X²⁶ | X²⁶ | |
| Adverse Event Assessment | X | X | X | X | X | X | X | X | X | X²⁸ |
| Concomitant Medications | X | X | X | X | X | X | X | X | X | X |
| Vital Status Assessment | | | | | | | | | | X²⁹ |

BP = blood pressure; ECG = electrocardiogram; EOI = end of infusion; EOT = End of Treatment; ETD = Early Treatment Discontinuation; HR = heart rate; NT-proBNP = N-terminal pro-brain natriuretic peptide; NYHA = New York Heart Association; PE = physical examination; SAE = serious adverse event; sFLCs = serum-free light chains; 6MWT = 6-minute walk test; SF-36v2 = Short Form-36 Version 2; WOCBP = women of childbearing potential.
¹The 28-day Screening period may be extended upon approval by the Medical Monitor. Individual test results that do not meet eligibility requirements may be repeated, with the exception of 6MWT; rescreening is allowed once per subject.
²Cycle 2-Days 8 and 22 and Cycle 3-Days 8, 15, and 22 bortezomib-containing chemotherapy should be administered by the Investigator at the study site if subject had significant toxicity; otherwise, it may be administered by local physician at the Investigator's discretion. See more details in Footnote 25.
³EOT/ETD Visit to occur 28 to 35 days after the last study drug administration.
⁴At the first Screening Visit, obtain comprehensive cardiac, hematologic, and oncologic medical history; additionally, for all other conditions, obtain relevant medical history for the past 5 years (including all major hospitalizations and surgeries), as well as the subject's current medical status. At the second Screening Visit, assess any changes/addition to medical history since the first Screening Visit.
⁵Results from mass spectrometry tissue typing, immunoelectron microscopy, gene sequencing, and/or ^{99m}Tc scintigraphy must be obtained prior to randomization to assess eligibility for subjects identified in inclusion criterion #5.
⁶At visits where the SF-36v2 Error! Reference source not found. is to be administered, the SF-36v2 needs to be administered prior to the performance of any other study assessments on that day.
⁷If an echocardiogram has been conducted within 90 days prior to Screening Day −28, it does not need to be repeated during Screening and the previous result can be used for eligibility. After Screening, perform echocardiograms every 6 months within 10 days prior to or on Day 1, within the study visit window; repeat at EOT/ETD if not performed within 60 days prior to visit.
⁸ECG to be performed in triplicate as follows: Month 1-Day 1: within 45 minutes before dosing and 1 hour (±15 min) post-EOI; and at every Day 1 visit for each month - within 45 minutes before dosing or any time on non-infusion days. Medications given for prophylaxis of chemotherapy-induced side effects should not be administered prior to completion of the post-infusion ECG.
⁹Complete PE includes height (Screening only), weight, and examination of the following: general appearance; head, ears, eyes, nose, and throat; neck; skin; cardiovascular system; respiratory system; gastrointestinal system; and nervous system. Assess macroglossia, submandibular nodes/fullness, adenopathy, ecchymoses, liver/spleen size (palpable +/−), ascites (+/−), and edema (which should be quantified on a scale of 0-4).
¹⁰Symptom-directed PE should be as clinically indicated and also include weight, and assessment of macroglossia, submandibular nodes/fullness, adenopathy, ecchymoses, liver/spleen size (palpable +/−), ascites (+/−), and edema (which should be quantified on a scale of 0-4).
¹¹The SARS-CoV-2 test should be conducted once within 14 days before dosing on Day 1, either during the first or second screening visit.
¹²Local laboratory results for hematology and chemistry will be used for subject management and should be reviewed for safety assessment prior to administration of chemotherapy but will not be collected in the electronic case report forms or the clinical database.
¹³Perform only if subject returns to study site for this visit.
¹⁴Use local laboratory for serum/urine pregnancy test within 24 hours prior to Month 1-Day 1 study drug administration.
¹⁵Obtain local laboratory serum/urine pregnancy test results prior to study drug administration.
¹⁶Collect central laboratory samples before 6MWT, if being performed on the same day.
¹⁷Collect prothrombin time/international normalized ratio and partial thromboplastin time at each time point.
¹⁸NT-proBNP should be drawn before conducting 6MWT if being performed on the same calendar day.
¹⁹Subjects should plan to be able to return to the same clinical site for each 6MWT from first Screening through Month 9 and at EOT/ETD. The postbaseline 6MWT may be administered on the same calendar day that study drug is administered (i.e., Months 3, 6, 9) as long as the NT-proBNP sample is drawn before conducting the 6MWT and the 6MWT is completed before initiation of the study drug infusion. Collect BP and HR pre- and post-6MWT administration.
²⁰The first Screening 6MWT must be performed between Days −28 and −5, at least 4 days apart from the second Screening 6MWT, which should be performed within 3 days prior to the Month 1-Day 1 visit (i.e., on Day −3 to Day −1).
²¹Randomization in the Interactive Voice and Web Response System (IXRS) can occur up to one day prior Month 1-Day 1 visit
²²All subjects are to receive 25 mg diphenhydramine (or equivalent dose of an H1 antihistamine) and 650 mg acetaminophen (or equivalent paracetamol dose in accordance with local practice) within 30 to 90 minutes prior to the start of infusion.
²³Vital signs include BP, HR, respiratory rate, and temperature; assess in the same position for all time points after the subject has been at rest for ≥5 minutes. Pre-dose assessments of vital signs should be performed after administration of premedication. Screening and non-infusion days: any time; Month 1-Day 1: within 30 minutes before dosing, halfway through infusion (i.e., approximately 60 minutes after the start of the infusion), immediately at EOI (±10 min), 0.5 hour (±10 min) post-EOI, and 1 hour (±10 min) post-EOI. All Other Months-Day 1: within 30 minutes before dosing, EOI (±10 min), and 1 hour (±10 min) post-EOI.
²⁴Subjects should be closely monitored for 90 (±10) minutes following completion of the study drug infusion. The Investigator may increase this standard monitoring time if deemed appropriate or per local standards. In the event of any clinical concerns or suspicious signs or symptoms after the infusion, the subject will remain under observation for as long as the Investigator deems it appropriate.
²⁵First-line chemotherapy must be a bortezomib-containing regimen, with bortezomib administered subcutaneously weekly, according to the approved prescribing information and local institutional practices. The initial first-line chemotherapy regimen may also include daratumumab at the discretion of the Investigator. Antiviral prophylaxis is required. When chemotherapy is administered on the same day as study drug, the chemotherapy must be administered AFTER the post-study drug infusion observation period. The number of first-line chemotherapy cycles and subsequent chemotherapy regimens will be administered per standard of care at the Investigator's discretion.
²⁶Bortezomib must be administered at the study site for Cycle 1-Days 1, 8, 15, and 22; Cycle 2-Days 1 and 15; and on Day 1 of subsequent cycles, after review of local laboratory results, study drug administration, and the post-study drug infusion observation period.
²⁷Cycle 2-Days 8 and 22, and Cycle 3-Days 8, 15, and 22, chemotherapy may be administered by local physician with a homecare visit by a Prothena-sponsored healthcare professional to the subject within 1 day prior to or pre-dose on the day of each bortezomib administration to obtain vital signs, blood samples for central laboratory testing, and bioanalytical samples (if applicable). If bortezomib is administered on a Monday, the homecare visit may occur on the previous Friday. If significant toxicity occurs during Cycle 1, subject should return to the study site for Cycle 2 and Cycle 3 visits until Investigator deems it appropriate for local administration.
²⁸New SAEs occurring beyond the EOT/ETD Visit or >28 days after the last administration of study drug, whichever is later, will be reported to the Sponsor or its designee only if, in the judgement of the Investigator, the SAE is associated with any protocol intervention (i.e., related to study procedure or previous study drug exposure).
²⁹For all subjects who are randomized and received a dose of study drug: conduct vital status assessment follow-up phone calls approximately 3 months after the subject's last visit and approximately every 3 months thereafter. If subjects discontinue study drug early but agree to return for study assessments. [symbol]
indicates data missing or illegible when filed

TABLE 2
Schedule of Events For Subjects Who Discontinue Study Drug Early
but Agree to Return For Assessments After the ETD Visit

| | Months 3, | Every Third Month | Every 3 |
|---|---|---|---|
| | 6, 9, 12 | After Month 12² | Months |
| | Day 1 | (e.g., Months 15, 18, | after Last |
| Assessments | (±5)¹ | 21) Day 1 (±5) | Visit |
| SF-36v2 Health | X | | |
| Survey³ | | | |
| Echocardiogram | Months 6 | | |
| | and 12⁴ | | |
| 12-lead Triplicate | X | | |
| ECG⁵ | | | |
| Symptom- | X | | |
| Directed PE⁶ | | | |
| NYHA Class | X | | |
| Local Laboratory | | | |
| Serum Pregnancy | X⁷ | | |
| (WOCBP) | | | |
| Central | | | |
| Laboratory⁸ | | | |
| Hematology & | X | | |
| Chemistry | | | |
| Amylase | X | | |
| Coagulation⁹ | X | | |
| Complements C₃, C₄ | X | | |
| Troponin T | X | | |
| NT-proBNP | X | | |
| Serum Free | X | | |
| Light Chains | | | |
| (sFLCs) | | | |
| Urinalysis | X | | |
| 6-Minute Walk | X | X¹⁰ | |
| Test (6MWT) | | | |
| Bioanalytical | | | |
| Laboratory | | | |
| Anti-NEOD001 | X | | |
| Antibody Sample¹¹ | | | |
| Vital Signs | X¹² | X¹⁰ | |
| Adverse Event | X¹³ | X^{10, 13} | |
| Assessment | | | |
| Concomitant | X | X¹⁰ | |
| Medications | | | |
| Health Status & | X¹⁴ | X^{10, 14} | X^{10, 13, 14} |
| Hospitalizations | | | |
| Vital Status Assessment | | | X¹⁵ |

BP = blood pressure; ECG = electrocardiogram; ETD = Early Treatment Discontinuation; HR = heart rate; NT proBNP = N terminal pro-brain natriuretic peptide; NYHA = New York Heart Association; PE = physical exam; RR = respiratory rate; 6MWT = 6-minute walk test; SF 36v2 = Short Form-36 Version 2; WOCBP = women of childbearing potential.
Table 2 Footnotes
¹If a subject discontinues study drug prior to the end of the study, but is willing to continue to participate in study visits, the subject should have an ETD Visit within 28-35 days after the last study drug administration and then have assessments performed every third month (i.e., Months 3, 6, 9, and 12, or whatever remains of these visits). All visits after the ETD Visit should occur on schedule, that is, at the time when the visit would have occurred had the subject remained on study drug.
²If subject is willing to return to the study site, otherwise, subjects will receive vital status.
³Administer SF-36v2 prior to the performance of any other study assessments on the day it is administered.
⁴Perform echocardiograms within 10 days prior to Day 1 of Months 6 and 12.
⁵ECG to be performed in triplicate.
⁶Symptom-directed PE should be as clinically indicated and also include weight, and assessment of macroglossia, submandibular nodes/fullness, adenopathy, ecchymoses, liver/spleen size (palpable +/−), ascites (+/−), and edema (which should be quantified on a scale of 0-4).
⁷Obtain local laboratory serum pregnancy test 90 (±5) days after the last study drug administration.
⁸Collect central laboratory samples before 6MWT, if being performed on the same day.
⁹Collect PT/INF and PTT at each time point.
¹⁰After Month 12, if the subject is willing to return to the study site, perform or collect the following every third month (e.g., Months 15, 18, 21): 6MWT (which includes BP and HR pre- and post-6MWT administration), adverse events, concomitant medications, overall health status, as well as details of any hospitalizations.
¹¹Collect if an earlier sample established the presence of anti-NEOD001 antibodies or if a subject discontinued treatment due to a suspected immunologic reaction.
¹²Collect BP, HR, RR, and temperature any time during visit.
¹³New SAEs occurring beyond the EOT/ETD Visit or >28 days after the last
administration of study drug, whichever is later, will be reported to the Sponsor or its designee only if, in the judgement of the Investigator, the SAE is associated with any protocol intervention (i.e., related to study procedure or previous study drug exposure).
¹⁴All hospitalizations and deaths occurring during this period need to be reported to the Sponsor or its designee.
¹⁵Conduct a vital status assessment approximately 3 months after the subject's last visit and approximately every 3 months thereafter.

### Example 2-Change in Global Longitudinal Strain (GLS)

Left ventricular (LV) global longitudinal strain (GLS), assessed by echocardiogram, is a measure of LV systolic function. GLS is a parameter that reflects the deformation of the left ventricle in the longitudinal direction which is linked to prognosis in many clinical conditions. GLS is reported as a negative value, and values for normal function are typically less than-20%. GLS was shown to be an independent predictor of overall survival in patients with AL amyloidosis-patients with lower (more negative) GLS values survived longer than patients with higher (less negative) GLS values. See, Buss 2012; Chuy 2020; Salinaro 2017.

GLS is evaluated in subjects from the AFFIRM-AL study (Example 1) according to procedures known to one of skill in the art. See, Buss 2012; Chuy 2020; Salinaro 2017. GLS is evaluated to determine change from baseline to Months 6, 12, and end of treatment.

Descriptive statistics for GLS may include actual values, change from baseline, and percent change from baseline in GLS by treatment group at Months 6, 12, and end of treatment in the ITT population.

Administration of birtamimab to Mayo stage IV AL amyloidosis patients reduces (improves) GLS. Reduction of GLS in Mayo stage IV AL amyloidosis patients administered birtamimab predicts reduced mortality (e.g., improved survival) of such patients.

Administration of birtamimab and daratumumab to Mayo stage IV AL amyloidosis patients reduces (improves) GLS. Reduction of GLS in Mayo stage IV AL amyloidosis patients administered birtamimab and daratumumab predicts reduced mortality (e.g., improved survival) of such patients.

### REFERENCES

Buss S J, Emami M, Mereles D, et al. Longitudinal left ventricular function for prediction of survival in systemic light-chain amyloidosis: incremental value compared with clinical and biochemical markers. J Am Coll Cardiol. 2012; 60 (12): 1067-76.
Chuy K L, Drill E, Yang J C, et al. Incremental value of global longitudinal strain for predicting survival in patients with advanced AL amyloidosis. JACC CardioOncol. 2020; 2 (2): 223-31.
Salinaro F, Meier-Ewert H K, Miller E J, et al. Longitudinal systolic strain, cardiac function improvement, and survival following treatment of light-chain (AL) cardiac amyloidosis. Eur Heart J Cardiovasc Imaging. 2017; 18 (9): 1057-64

1. A method of treating a patient having AL amyloidosis, comprising:
administering an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) to the patient; and
5 to 90 minutes after completion of administering the antibody, administering a therapeutically effective dosage of daratumumab to the patient,
wherein the patient has one or more of the following:
(a) Mayo Stage IV AL amyloidosis,
(b) a 6 minute walk distance (6MWT)≥30 meters and ≤550 meters,
(c) a 6MWT≥150 meters and EF>50%,
(d) Mayo Stage IV and an EF>50%,
(e) Mayo Stage IV and a 6MWT≥30 meters and ≤550 meters,
(f) Mayo Stage IV and a 6MWT≥150 meters and EF>50%,
(g) has cardiac involvement;
(h) has Mayo Stage IV AL amyloidosis and has cardiac involvement; or
(i) has Mayo Stage IV AL amyloidosis, cardiac involvement, and a 6MWT≥30 meters and ≤550 meters.

2. The method of claim 1, wherein the patient has Mayo Stage IV AL amyloidosis.

3. The method of claim 1, wherein the patient has a 6MWT≥150 meters and an EF>50%.

4. The method of claim 1, wherein the patient has a 6MWT≥30 meters and ≤ 550 meters.

5. The method of claim 4, wherein the patient has an EF>50%.

6. The method of claim 5, wherein the patient has a 6MWT≥150 meters.

7. The method of claim 1, wherein the patient has cardiac involvement.

8. The method of claim 1, wherein the patient has Mayo Stage IV AL amyloidosis and has cardiac involvement.

9. The method of claim 1, wherein the patient has Mayo Stage IV AL amyloidosis, cardiac involvement, and a 6MWT≥30 meters and ≤550 meters.

10-19. (canceled)

20. A method of treating a patient having Mayo Stage IV AL amyloidosis and having NYHA class III or NYHA class IV, the method comprising:
administering to the patient a therapeutically effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105) amount; and
5 to 90 minutes after completion of administering the antibody, administering a therapeutically effective dosage of daratumumab to the patient, wherein the patient has one or more of the following:
(a) a 6 minute walk distance (6MWT)≥30 meters and ≤550 meters,
(b) a 6MWT≥150 meters and EF>50%,
(c) Mayo Stage IV and an EF>50%,
(d) Mayo Stage IV and a 6MWT≥30 meters and ≤550 meters,
(e) Mayo Stage IV and a 6MWT≥150 meters and EF>50%,
(f) has cardiac involvement;
(g) has Mayo Stage IV AL amyloidosis and has cardiac involvement; or
(h) has Mayo Stage IV AL amyloidosis, cardiac involvement, and a 6MWT≥30 meters and ≤550 meters.

2-171. (canceled)

172. A method of reducing the risk of mortality in a patient with AL amyloidosis comprising:
administering to the patient an effective dosage of an antibody which competes for binding to human amyloid A peptide or human kappa or lambda light chain immunoglobulin with 2A4 (ATCC Accession Number PTA-9662) or 7D8 (ATCC Accession Number PTA-9468), or competes for binding to kappa light chain immunoglobulin with 11-1F4 (ATCC Accession Number PTA-105), 6MWT and
5 to 90 minutes after completion of administering the antibody, administering a therapeutically effective amount of daratumumab to the patient, wherein the patient has one or more of the following:
(a) Mayo Stage IV AL amyloidosis,
(b) a 6 minute walk distance (6MWT)≥30 meters and ≤550 meters,
(c) a 6MWT≥150 meters and EF>50%,
(d) Mayo Stage IV and an EF>50%,
(e) Mayo Stage IV and a 6MWT≥30 meters and ≤550 meters,
(f) Mayo Stage IV and a 6MWT≥150 meters and EF>50%,
(g) has cardiac involvement;
(h) has Mayo Stage IV AL amyloidosis and has cardiac involvement; or
(i) has Mayo Stage IV AL amyloidosis, cardiac involvement, and a 6MWT≥30 meters and ≤550 meters.

173. The method of claim 172, wherein the risk of mortality is of all-cause mortality.

174-180. (canceled)

181. The method of claim 172, wherein the risk of mortality is of cardiac mortality.

182-283. (canceled)

284. The method of claim 1, wherein the antibody comprises a light chain variable region comprising three complementarity determining regions set forth as SEQ ID NOs: 3, 4 and 5, or SEQ ID NOs: 16, 17, and 18, and a heavy chain variable region comprising three complementarity determining regions set forth as SEQ ID NOs: 6, 7 and 8, or SEQ ID NOs: 19, 20, and 21.

285-286. (canceled)

287. The method of claim 1, wherein the light chain variable region comprises the amino acid sequence set forth as SEQ ID NO: 1 or 14, and the heavy chain variable region comprises the amino acid sequence set forth as SEQ ID NO: 2 or 15.

288-289. (canceled)

290. The method of claim 1, wherein the patient previously received or concomitantly receives treatment with melphalan, prednisone, dexamethasone, bortezomib, cyclophosphamide, lenalidomide, doxorubicin, or a combination thereof.

291-293. (canceled)

294. The method of claim 1, wherein the antibody is birtamimab.

295-297. (canceled)

298. The method of claim 1, wherein the therapeutically effective dosage of the antibody is from about 0.5 mg/kg to about 30 mg/kg and the antibody is administered intravenously or subcutaneously at a frequency of from about weekly to about quarterly.

299. The method of claim 298 wherein the therapeutically effective dosage of the antibody is present at a concentration of about 50 mg/mL.

300. (canceled)

301. The method of claim 298, wherein the therapeutically effective dosage is about 24 mg/kg and the antibody is administered intravenously every 28 days.

302-332. (canceled)
